(19) United States
(12) Patent Application Publication
(10) Pub. No.: US 2022/0101962 A1
(43) Pub. Date: Mar. 31, 2022

(54) ENABLING DISTRIBUTED SEMANTIC MASHUP

(71) Applicant: CONVIDA WIRELESS, LLC, Wilmington, DE (US)

(72) Inventors: Mingming GUO, Maryland Heights, MO (US); Xu LI, Plainsboro, NJ (US); Chonggang WANG, Princeton, NJ (US); Quang LY, North Wales, PA (US); Lu LIU, Conshohocken, PA (US)

(21) Appl. No.: 17/421,844

(22) PCT Filed: Jan. 10, 2020
(86) PCT No.: PCT/US2020/013003

§ 371 Date: Jul. 9, 2021

Related U.S. Application Data
(60) Provisional application No. 62/791,338, filed on Jan. 11, 2019.

(51) Int. Cl. G16H 10/60; G06F 40/30

(52) U.S. Cl. CPC G16H 10/60; G06F 40/30

(57) ABSTRACT

Distributed Semantic Mashup Service (DSMS) architecture may address the limitations of centralized mashup service. DSMS may leverage a group of SMS Hosts to conduct mashup operations in a distributed manner, such as each involved SMS Host conducts certain mashup operations locally on its own, and finally conducts mashup operation on the Master SMS Host based on these distributed Child Semantic Mashup Instances s to generate more advanced Hierarchical Semantic Mashup Instances to be returned to SMS Requestors.

### CROSS-REFERENCE TO RELATED APPLICATION

This application claims the benefit of U.S. Provisional Patent Application No. 62/791,338, filed Jan. 11, 2019, which is hereby incorporated by reference in its entirety.

### BACKGROUND

The oneM2M standard under development defines a Service Layer called “Common Service Entity (CSE)”. The purpose of the Service Layer is to provide “horizontal” services that can be utilized by different “vertical” M2M systems and applications. The CSE supports four reference points as shown in FIG. 1. The Mca reference point interfaces with the Application Entity (AE). The Mcc reference point interfaces with another CSE within the same service provider domain and the Mcc′ reference point interfaces with another CSE in a different service provider domain. The Mcn reference point interfaces with the underlying network service entity (NSE). An NSE provides underlying network services to the CSEs, such as device management, location services and device triggering. CSE contains multiple logical functions called “Common Service Functions (CSFs)”, such as “Discovery” and “Data Management & Repository”. FIG. 2 illustrates some of the CSFs defined by oneM2M. The oneM2M architecture enables the following types of Nodes as shown in FIG. 1:
Application Service Node (ASN): An ASN is a Node that contains one CSE and contains at least one Application Entity (AE). Example of physical mapping: an ASN could reside in an M2M Device.
Application Dedicated Node (ADN): An ADN is a Node that contains at least one AE and does not contain a CSE. There may be zero or more ADNs in the Field Domain of the oneM2M System. Example of physical mapping: an Application Dedicated Node could reside in a constrained M2M Device.
Middle Node (MN): A MN is a Node that contains one CSE and contains zero or more AEs. There may be zero or more MNs in the Field Domain of the oneM2M System. Example of physical mapping: a MN could reside in an M2M Gateway.
Infrastructure Node (IN): An IN is a Node that contains one CSE and contains zero or more AEs. There is exactly one IN in the Infrastructure Domain per oneM2M Service Provider. A CSE in an IN may contain CSE functions not applicable to other node types. Example of physical mapping: an IN could reside in an M2M Service Infrastructure.
Non-oneM2M Node (NoDN): A non-oneM2M Node is a Node that does not contain oneM2M Entities (neither AEs nor CSEs). Such Nodes represent devices attached to the oneM2M system for interworking purposes, including management.

oneM2M specifies Semantic Mashup Function (SMF) which is responsible for collecting the data inputs from data sources hosted on Resource Hosts (RHs) and mashing them up to generate the mashup result based on a certain business logic. In the context of oneM2M, SMF is a Common Service Function, which can be located in a SMS Host. In order to leverage semantic mashup, a Mashup Requestor (MR) initiates a mashup request to Semantic Mashup Function for a certain need. In the context of oneM2M, an AE or a CSE can be an MR.

### SUMMARY

The conventional centralized semantic mashup methods in M2M/IoT service layer need to collect all related input data or resources at a single place before mashing them up and thus they are not sufficient to address the problems in many emerging use cases (e.g. Smart eHealth, etc.). The mashup basically takes related data or resources as input and outputs some knowledge or valuable information according to certain mashup operation logic. In these emerging use cases, the clients or applications (e.g., users) may not be able to get their services in a more powerful/useful way or may be even impossible to get the enriched knowledge only on their subscribed M2M/IoT servers due to the fact that many original resources residing on other related M2M/IoT servers are only available and retrievable to local access due to security/privacy consideration. This disclosure discloses new mechanisms to enable distributed semantic mashup services in M2M/IoT service layer in order to enhance system capability, to enable the knowledge extension, and to improve service quality.

The ideas being disclosed in this disclosure include:

Distributed Semantic Mashup Service (DSMS) architecture to address the limitations of centralized mashup service. DSMS leverages a group of SMS Hosts to conduct mashup operations in a distributed manner (e.g. each involved SMS Host will conduct certain mashup operations locally on its own), and finally conducts mashup operation on the Master SMS Host based on these distributed Child SMIs to generate more advanced Hierarchical SMI to be returned to SMS Requestors.

SMS Indication and SMS Discovery. The structure of an SMS Capability Indication is specified, and the SMS Indication and SMS Discovery processes are disclosed to enable the SMS capability discovery, interoperability and usage.

Hierarchical SMJP Association and Generation. The new attribute named related SMJPs is disclosed to model the Parent-Child relationship among different SMJPs. The Hierarchical SMJP association and modification processes are disclosed to enable the dynamic SMJP association and deletion among different SMJPs.

Distributed semantic mashup with sequential execution dependency for generating Hierarchical SMIs as a working instance of a specific hierarchical SMJP. The DSMS with sequential execution dependency is disclosed to enable the sequential execution when the Child SMJPs of a Hierarchical SMJP have dependencies.

Distributed semantic mashup with parallel execution without dependency for generating Hierarchical SMIs. The DSMS with parallel execution is disclosed to enable the parallel execution when the Child SMJPs of a Hierarchical SMJP have no dependencies.

Distributed semantic mashup without SMJP association using semantic discovery. The DSMS without SMJP association is disclosed to enable the discovery and mashup of Child SMIs that satisfying the requested SMJP on Master SMS Host to generate the Hierarchical SMI for SMS Requestors.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not constrained to limitations that solve any or all disadvantages noted in any part of this disclosure.

### BRIEF DESCRIPTION OF THE DRAWINGS

A more detailed understanding may be had from the following description, given by way of example in conjunction with the accompanying drawings wherein:

FIG. 1 illustrates an exemplary oneM2M Architecture;

FIG. 2 illustrates an exemplary oneM2M Common Service Functions;

FIG. 3 illustrates an exemplary Smart eHealth Use Case;

FIG. 4 illustrates an exemplary Semantic Mashup Problem due to Data Privacy or Other Issues;

FIG. 5 illustrates an exemplary Overall Procedure of Distributed Semantic Mashup Service;

FIG. 6 illustrates an exemplary Hierarchical SMJP and SMI Relationship Graph;

FIG. 7 illustrates exemplary Functional Operations of Distributed Semantic Mashup;

FIG. 8 illustrates exemplary Elements of smsIndication Parameter;

FIG. 9 illustrates an exemplary SMS Indication during the Service Layer Registration Process;

FIG. 10 illustrates an exemplary SMS Host Announce its SMS Capability to Service Layer Node;

FIG. 11 illustrates an exemplary SMS Host Discovery Process Using smsFilter in the Request by Requestor;

FIG. 12 illustrates an exemplary SMS Host Notifies its Capability Update to Other SMS Hosts;

FIG. 13 illustrates an exemplary Example of SMJP's Representation with relatedSMJPs Attribute in Smart eHealth;

FIG. 14 illustrates an exemplary Example of SMJPs' Relationship Modeling in Smart eHealth on Different SMS Hosts;

FIG. 15 illustrates an exemplary SMJP2's Output Parameters Match the Required Parameters of memberFilter in SMJP1;

FIG. 16 illustrates an exemplary SMJP2's Input Parameters (if exists) is a Set/Subset of SMJP1's Input Parameters;

FIG. 17 illustrates an exemplary Process for Building Association between Two SMJPs Located on Two SMS Hosts on the Parent SMS Host Side;

FIG. 18 illustrates exemplary relatedSMJPs Attribute in SMJP1;

FIG. 19 illustrates exemplary RDF Triples that Modelling the Parent-Child Relationship;

FIG. 20 illustrates exemplary relatedSMJPs Attribute in SMJP2;

FIG. 21 illustrates an exemplary Process for Building Association between Two SMJPs Located on Two SMS Hosts on the Child Host Side;

FIG. 22 illustrates an exemplary Process for Building a New SMJP based on other SMJPs;

FIG. 23 illustrates exemplary relatedSMJPs in SMJP1 (New SMJP);

FIG. 24 illustrates exemplary relatedSMJPs in SMJP3 (Child SMJP);

FIG. 25 illustrates an exemplary Process for Hierarchical SMJP Modification and Deletion;

FIG. 26 illustrates exemplary relatedSMJPs in SMJP1 on SMS Host 121 after Modification;

FIG. 27 illustrates an exemplary Generate Hierarchical SMI with Sequential Execution Dependency;

FIG. 28 illustrates exemplary No Dependency between SMJP1 and SMJP2 in <relatedSMJPs> of SMJP1;

FIG. 29 illustrates an exemplary Generation of Hierarchical SMI with Parallel Execution without Dependency;

FIG. 30 illustrates an exemplary Generation of Hierarchical SMI without SMJP Association;

FIG. 31 illustrates an exemplary Distributed Semantic Mashup Service in the oneM2M Architecture;

FIG. 32 illustrates an exemplary Enhanced Semantic Mashup Job Profile Ontology;

FIG. 33 illustrates an exemplary Overall Procedure for Distributed Semantic Mashup in oneM2M Architecture;

FIG. 34 illustrates an exemplary User Interface for SMS Hosts for Distributed Semantic Mashup Service;

FIG. 35 illustrates an exemplary display that may be generated based on the methods and systems discussed herein;

FIG. 36A illustrates an exemplary machine-to-machine (M2M) or Internet of Things (IoT) communication system in which the disclosed subject matter may be implemented;

FIG. 36B illustrates an exemplary architecture that may be used within the M2M/IoT communications system illustrated in FIG. 36A;

FIG. 36C illustrates an exemplary M2M/IoT terminal or gateway device that may be used within the communications system illustrated in FIG. 36A; and

FIG. 36D illustrates an exemplary computing system in which aspects of the communication system of FIG. 36A.

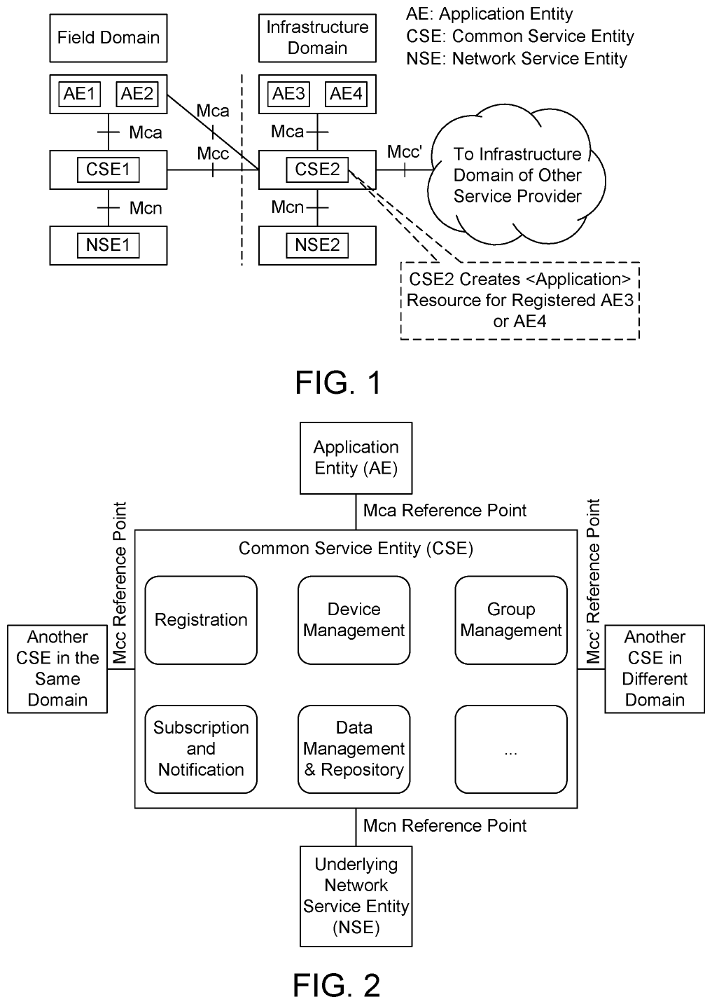

### DETAILED DESCRIPTION

Semantic Mashup Function (SMF) may be implemented based on the following components or resources which may be implemented using Semantic Mashup Job Profile (SMJP) or Semantic Mashup Instance (SMI). Semantic Mashup Job Profile (SMJP): Each specific semantic mashup application has a corresponding SMJP, which not only provides functionality or interaction details for external entities to discover (e.g. MRs), but also defines the internal working details regarding how to realize this mashup application (e.g. the criteria of how to select the qualified data sources as well as the definition of mashup function). Semantic Mashup Instance (SMI): Once an MR identifies a desired SMJP (which can be analogous to a “job description”, but not a real job), it can ask SMF to initialize a real mashup process, which corresponds to a “working instance” of this SMJP and is referred to as a Semantic Mashup Instance (SMI). In order to do so, the SMF may inject the corresponding SMJP into the Mashup Engine of SMF for the SMI instantiation, during which the engine may be involved in: 1) Identifying the qualified data sources according to the data source criteria as defined in the SMJP; 2) Collecting data inputs from those identified data sources; 3) Mashing up the collected inputs by applying mashup functions as defined in the SMJP, and 4) finally deriving the mashup result.

An SMF usually involves several different tasks or operations for realizing a complete semantic mashup process. These operations include: SMJP discovery, SMI creation, mashup member identification, data input collection and mashup result generation, or SMI discovery and re-use.

Overall, the MR may be a logical node requesting a specific semantic mashup operation, which is to be done by the SMF. An SMJP is a template-based job specification to describe a specific SMF, which defines the inputs or outputs as well as the processing logic of the mashup job. In the meantime, the SMJP library is a repository hosted by the SMS Host, where MRs can look for the desired SMJP based on their needs. When the SMS Host needs to conduct a specific semantic mashup operation, a SMI may be created by the SMS Host, which may represent a “working instance” that executes the corresponding SMJP of the required mashup service (which may imply that the same SMJP could be used to create multiple SMIs and each of them represents a separate SMF instance). For an SMI, the SMS Host may follow the details as specified in SMJP for collecting data inputs and mashing up the data inputs according to the mashup operation logics, and finally producing the mashup result.

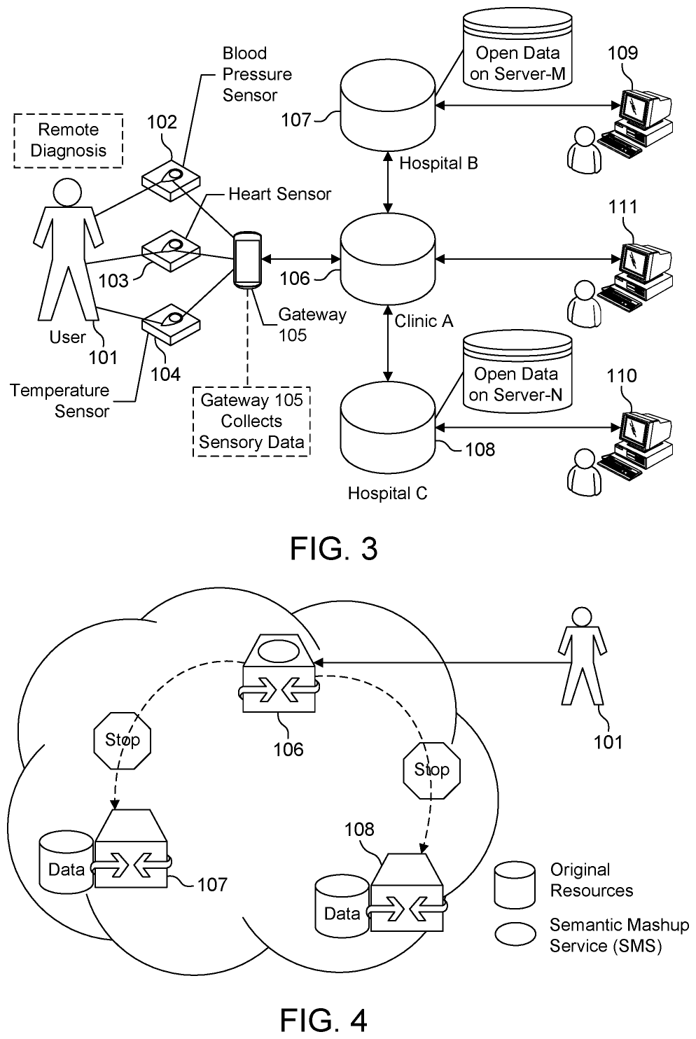

In a Smart eHealth scenario, a health diagnosis application is shown in FIG. 3. FIG. 3 is an example in which a user 101 wears a group of sensors to record and monitor their respective health conditions. For example, three types of sensors may be included such as blood pressure sensor 102, heartbeat sensor 103, or temperature sensor 104. Each of them may be registered with gateway 105, which may me a mobile phone of user 101. Gateway 105 may be registered to server 106 in Clinic A. Server 106 is also registered to server 107 in Hospital B and server 108 in Hospital C, respectively. Each server of FIG. 3 may have certain data privacy or other regulation policies and may not allow other servers to directly access or retrieve its local data. However, each server may receive requests from other servers and may provide some aggregated data (e.g., anonymized data) to the requestor without violating data privacy or other regulation policies. Server 106 may not be able to directly access the symptom data at server 107 or server 108. Server 108 data may be private for only Hospital C. Server 107 data may be private for only Hospital B.

Consider the case that user 101 has some unusual symptoms as observed by the wearable sensors. Gateway 105 might give an alert to u user 101 about the case to attract the attention of user 101. User 101 may also input additional description information about the symptoms like how user 101 feels through the phone. Then, user 101 may decide to send a diagnosis request to Server 106 through gateway 105, which may be a mobile phone of user 101 in this case. Since Clinic A has limited expertise and patient dataset on server 106, as we have assumed for this use case, it may not have the capability to provide an accurate diagnosis result. Upon receiving the diagnosis request from the gateway 105, server 106 may decide to mash up data from different sources (e.g. from other Hospital B or Hospital C) to get the clues for the unusual symptoms, in case of its limited data set and expertise. In fact, there might be thousands of patient's health information records distributed on each of the external servers. In this scenario, it may not be possible or may be inefficient for server 106 to retrieve the private sources from server 107 and server 108 108. In an efficient way, server 106 may send a diagnosis request with the patient's information (e.g., the symptoms) to server 107 or server 108 108, respectively, in order to let them locally mashup related patient data leveraging edge computing at server 107 and server 108 and get the diagnosis results. All the diagnosis results from server 107 and server 108 will be returned to server 106 for final mashup operation. After conducting the final mashup operation, the final diagnosis result (e.g., a potential disease) will be returned from server 106 to user 101. Server 107 may mashup the patient records to generate the disease results with anonymized patient records at 109. Server 108 may mashup the patient records to generate the disease results with anonymized patient records at 110. Server 106 may mashup the diagnosis results from server 107 and server 108 to provide a final diagnosis result to user 101.

Considering the smart health scenario, requests from user 101 (e.g., the requesting device or application) to a centralized mashup process (e.g., on server 106) may require hosting server access to the original data sources from different gateways or servers (e.g. server 107, server 108). In fact, the patient data on server 107 in Hospital B may not be allowed to be retrieved by external servers. On the other hand, server 107 in Hospital B may be able to help the requestor diagnose the patient's symptoms based on its private data records running its own mashup service. This issue may be encountered when access and retrieval of original data sources necessary for a centralized mashup is not available or prohibited, for example due to data privacy, deployment requirements, etc. As shown in FIG. 4, for the centralized approach, the original data sources on those protected servers, such as M2M server 107 and M2M server 108, may become unavailable for the mashup process on the mashup server such as M2M server 106. In some cases, the original data is not available or not allowed to be transmitted or even accessed due to privacy or other issues. As a result, conventionally, server 106 may not accomplish mashup operations. In other cases, there are only basic mashup services available and re-usable, but users request advanced mashup services. As a result, conventional centralized mashup services become limited or non-operational, since it needs to retrieve original resources (e.g., all original resources) from resource hosts. For this scenario, an issue is how to enable efficient semantic mashup service over distributed data (e.g., original resources), which is distributed in different places other than a centralized SMS host.

The centralized semantic mashup methods in M2M/IoT service layer need to collect all related input data or resources at a single place before mashing them up and thus they are not sufficient to address the problems in many emerging use cases (e.g., Smart eHealth, etc.). Here the mashup basically takes related data or resources at input and outputs some knowledge or valuable information according to certain mashup operation logic. In these emerging use cases, the users (e.g., applications) may not be able to get their services in a more useful way or may be even impossible to get the enriched knowledge only on their subscribed M2M/IoT servers due to the fact that many original resources residing on other related M2M/IoT servers are only available and retrievable to local access due to privacy consideration. Disclosed herein are mechanisms to enable distributed semantic mashup services in M2M/IoT service layer which may enhance system capability, to enable the knowledge extension, and to improve service quality.

There is a plurality of subject matter disclosed herein with regard to enabling distributed semantic mashup in an IoT service layer. First, disclosed herein is distributed semantic mashup service (DSMS) architecture that may address limitations of centralized mashup service. DSMS may leverage a group of SMS Hosts to conduct mashup operations in a distributed manner (e.g., each involved SMS Host may conduct certain mashup operations locally on its own), and then may conduct mashup operation on a master SMS host based on these distributed child SMIs to generate more advanced hierarchical SMI to be returned to SMS requestors. An SMS requestor may be a logical or physical entity that requests and uses SMSs. An SMS requestor may discover (or even create) semantic mashup job profiles (SMJPs), create/discover/access SMIs according to certain SMJPs, and access SMIs. For example, a oneM2M CSE/AE may be an SMS requestor.

Secondly, disclosed herein are methods or systems for SMS indication or SMS Discovery. The structure of an SMS capability indication may be specified, and the SMS indication or SMS Discovery processes may enable the SMS capability discovery, interoperability, or usage. Thirdly, methods or systems for hierarchical SMJP association or generation are disclosed. An attribute that may be named relatedSMJPs may model the Parent-Child relationship among different SMJPs. The Hierarchical SMJP association and modification processes may enable the dynamic SMJP association or deletion among different SMJPs.

Fourthly, disclosed herein are methods or systems for distributed semantic mashup with sequential execution dependency for generating hierarchical SMIs as a working instance of a specific hierarchical SMJP. The DSMS with sequential execution dependency may enable the sequential execution when the child SMJPs of a hierarchical SMJP have dependencies. Fifthly, disclosed herein are methods or systems for distributed semantic mashup with parallel execution without dependency for generating hierarchical SMIs. The DSMS with parallel execution may enable the parallel execution when the child SMJPs of a hierarchical SMJP have no dependencies. Sixthly, disclosed herein are methods or systems for distributed semantic mashup without SMJP association using semantic discovery. The DSMS without SMJP association is disclosed to enable the discovery and mashup of child SMIs that satisfying the requested SMJP on Master SMS Host to generate the hierarchical SMI for SMS requestors.

The disclosed Distributed Semantic Mashup Service (DSMS) provides distributed mashup among multiple SMS Hosts where their original resources have privacy/regulation/size constraints. In conventional service layers like oneM2M, SMS Hosts are independent of each other and have little to no interaction among them regarding their SMS capabilities. In the disclosed DSMS, a SMS host may provide mashup services not only to SMS Requestors, but also to other SMS hosts. In other words, a SMS host may leverage mashup services from other SMS hosts to generate more advanced mashup services. This may be accomplished by enabling SMS capability discovery between several SMS hosts and enabling hierarchical SMJP and SMI associations. The related terms have been defined in Table 5 below. Note SMS Requestor introduced in this disclosure may have similar or the same role as a mashup requestor (MR) in oneM2M. A SMS host may host a list of support SMSs, meaning that not all SMSs must be deployed on a given SMS host. A specific SMS may support a list of SMJPs; even if a SMS Host is running or otherwise providing a SMS, such a SMS can only support a list of specific SMJPs. SMS requestor may be a logical or physical entity, which requests and uses (e.g., consumes) SMSs. An SMS Requestor may discover (or even create) SMJPs, create/discover/access SMIs according to certain SMJPs, and access SMIs. For example, an oneM2M CSE/AE may be an SMS Requestor.

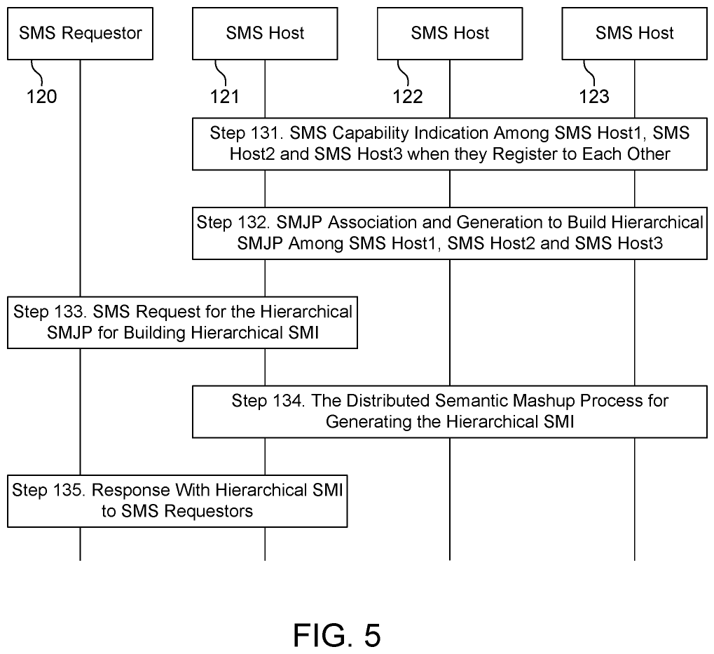

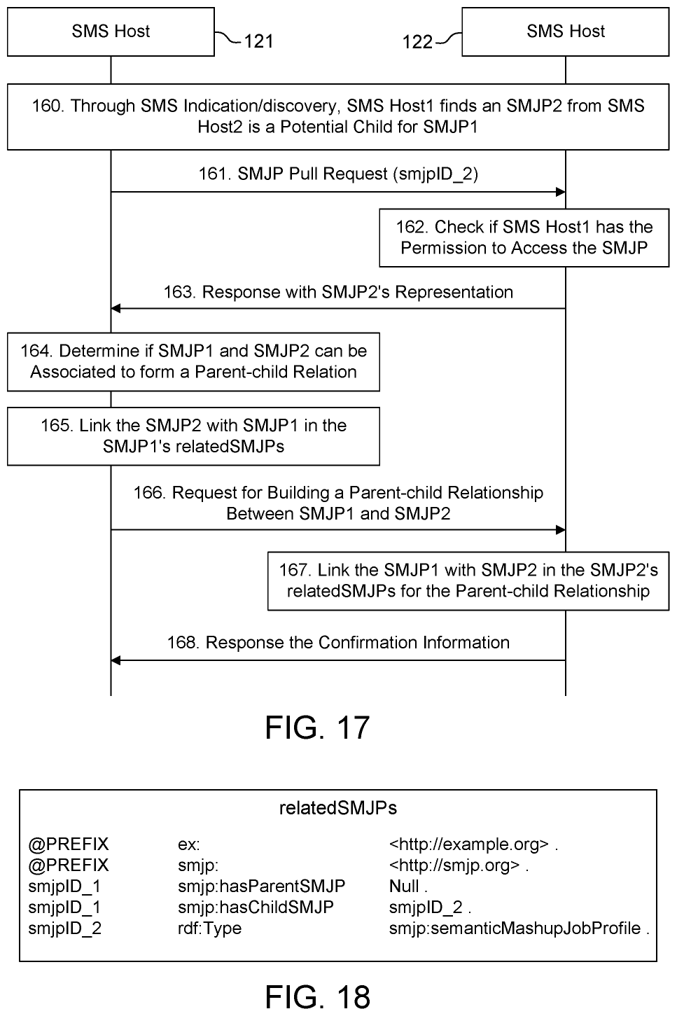

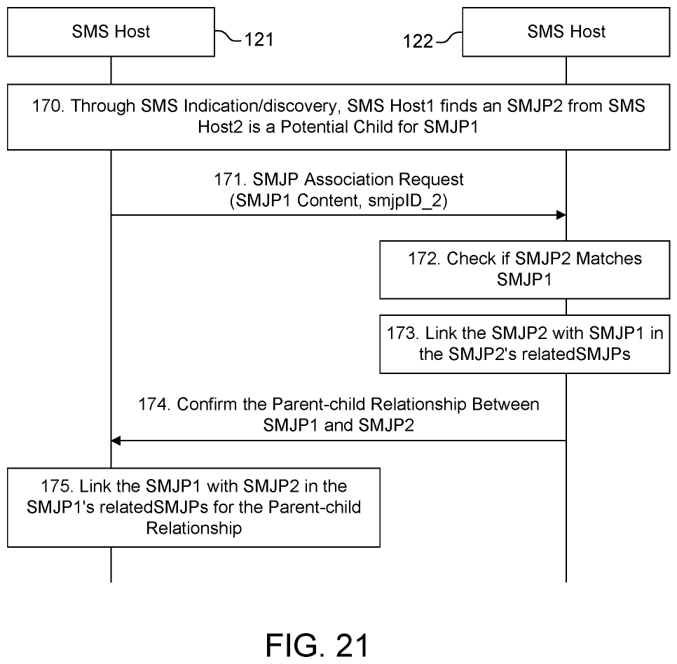

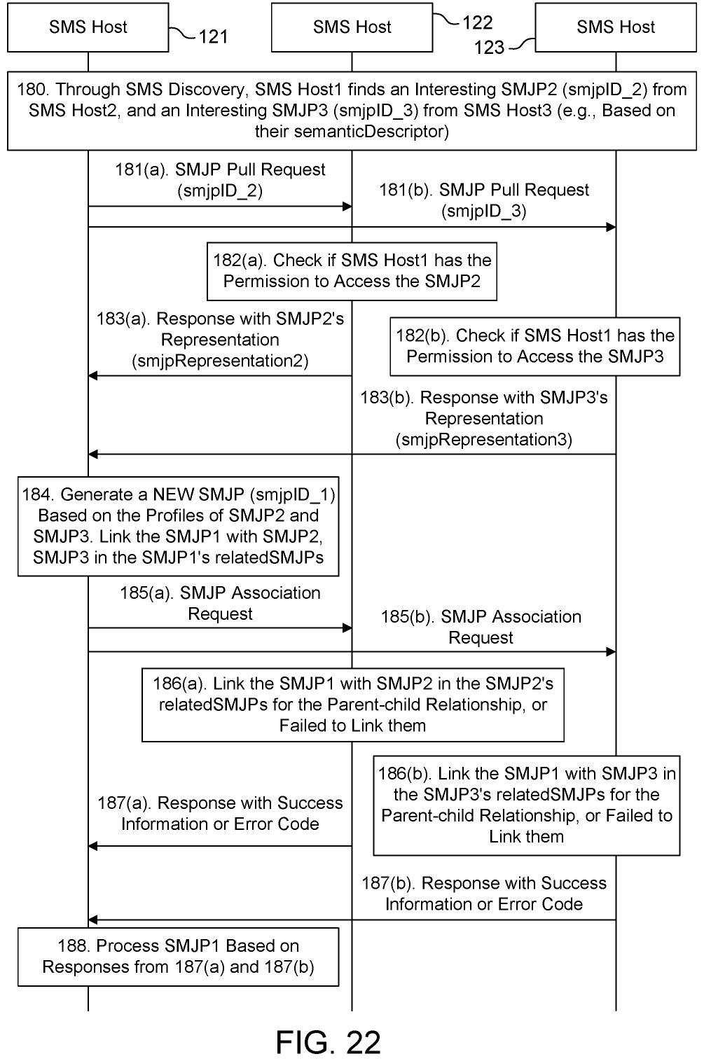

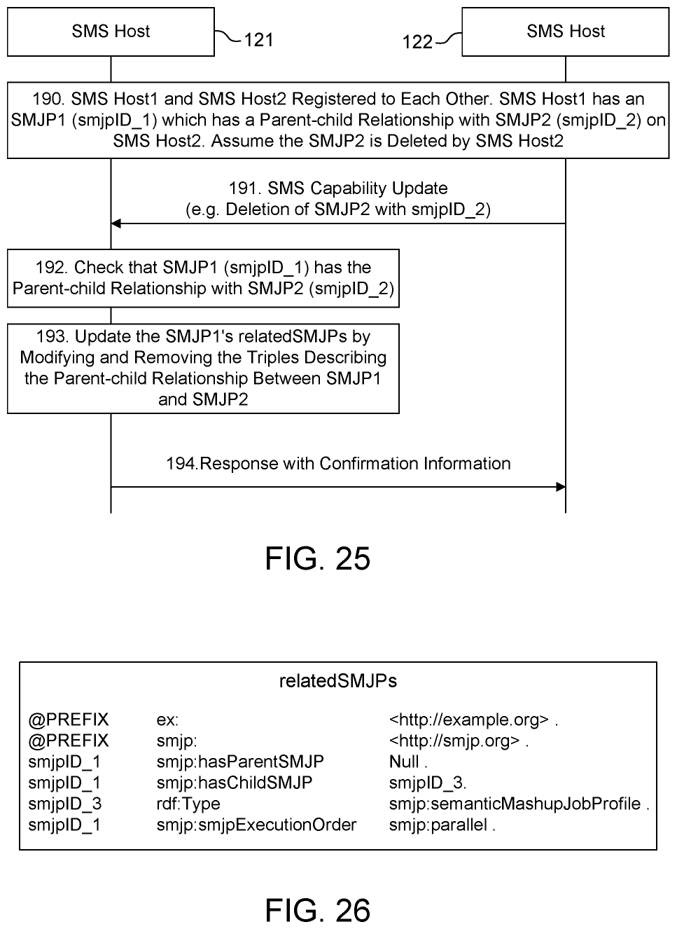

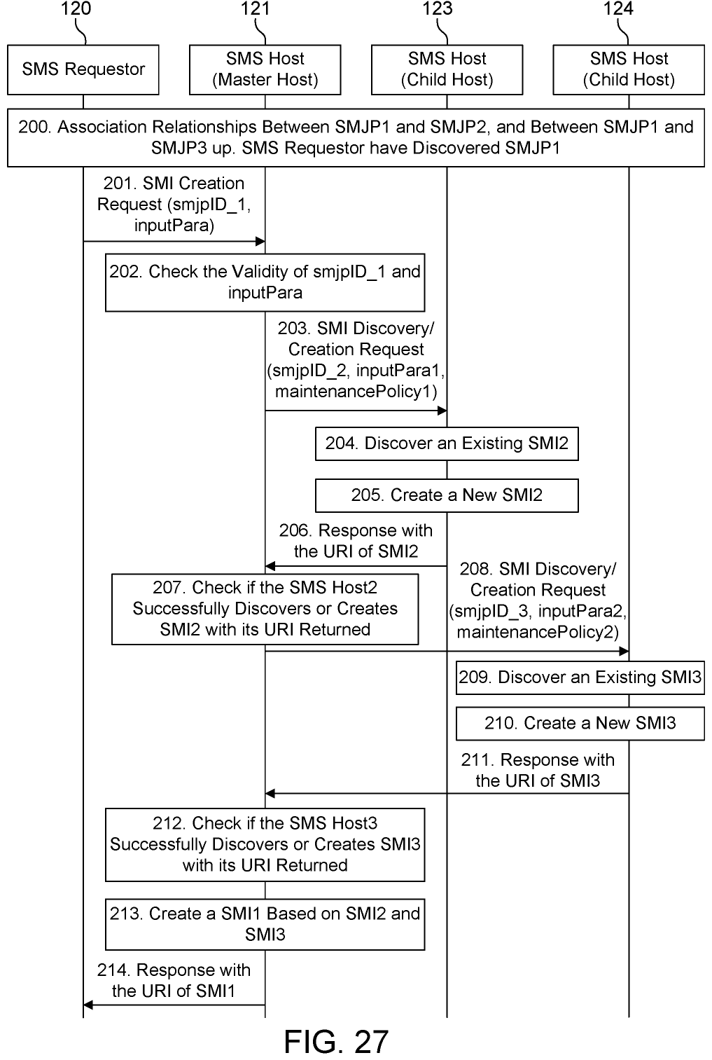

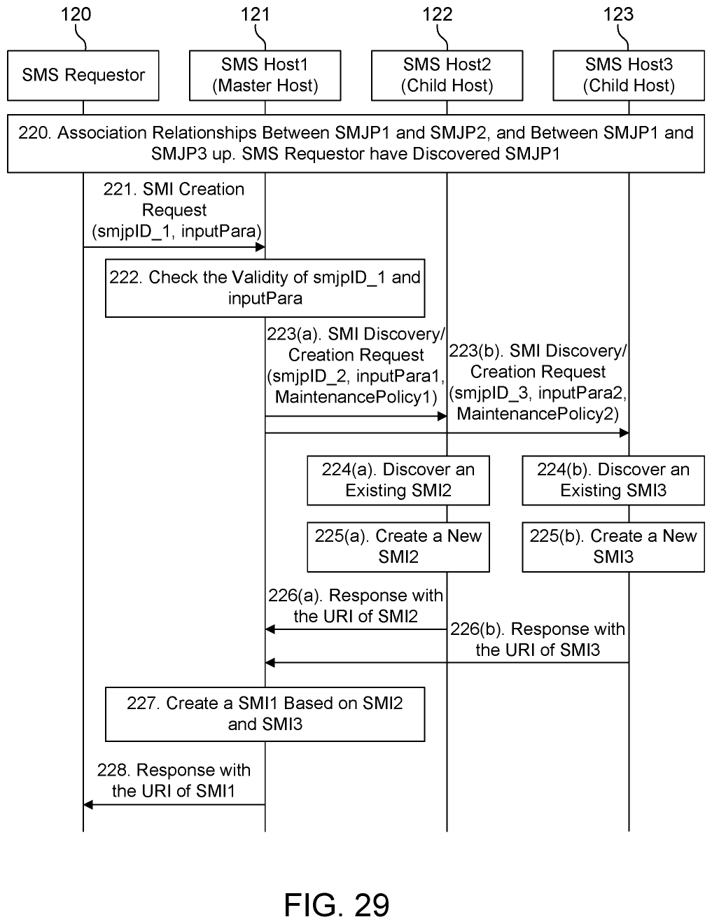

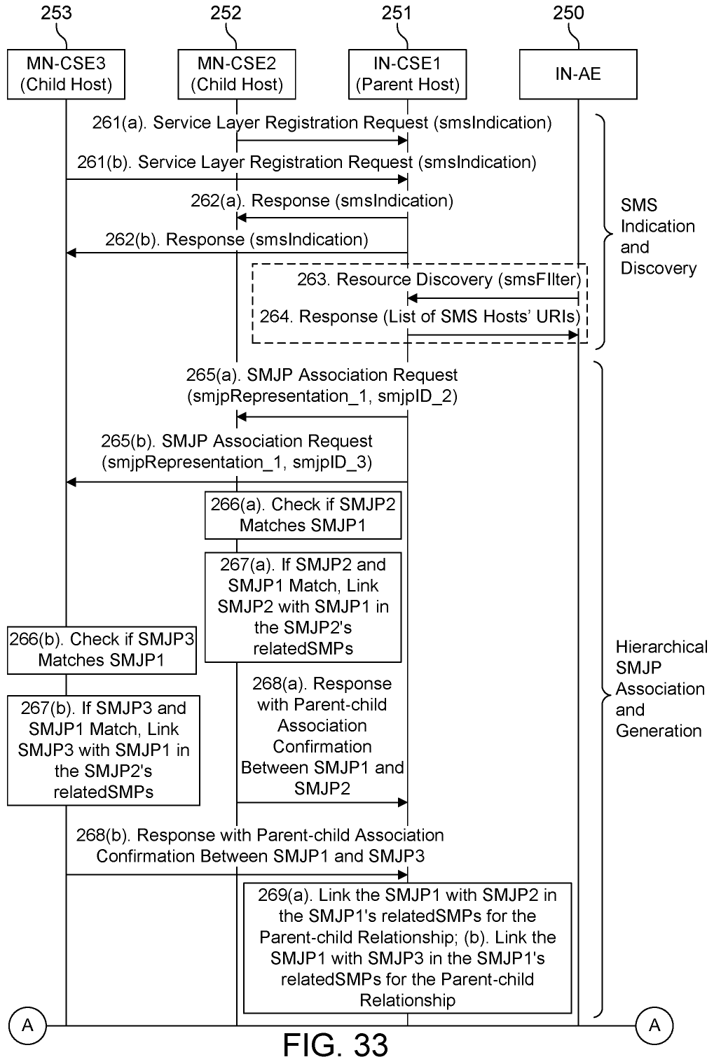

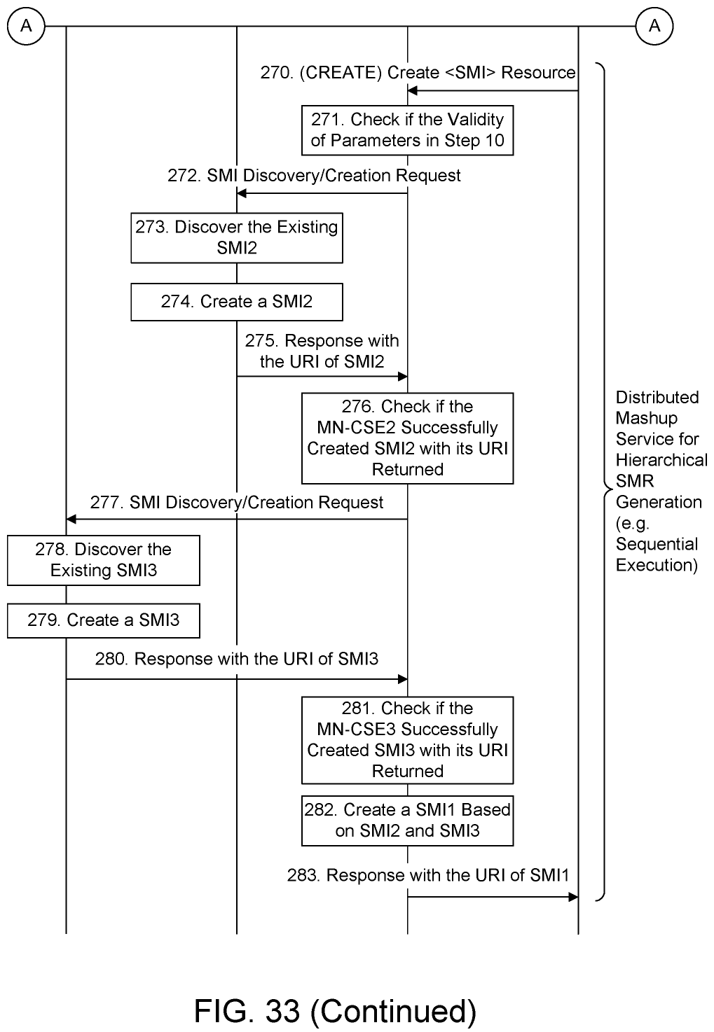

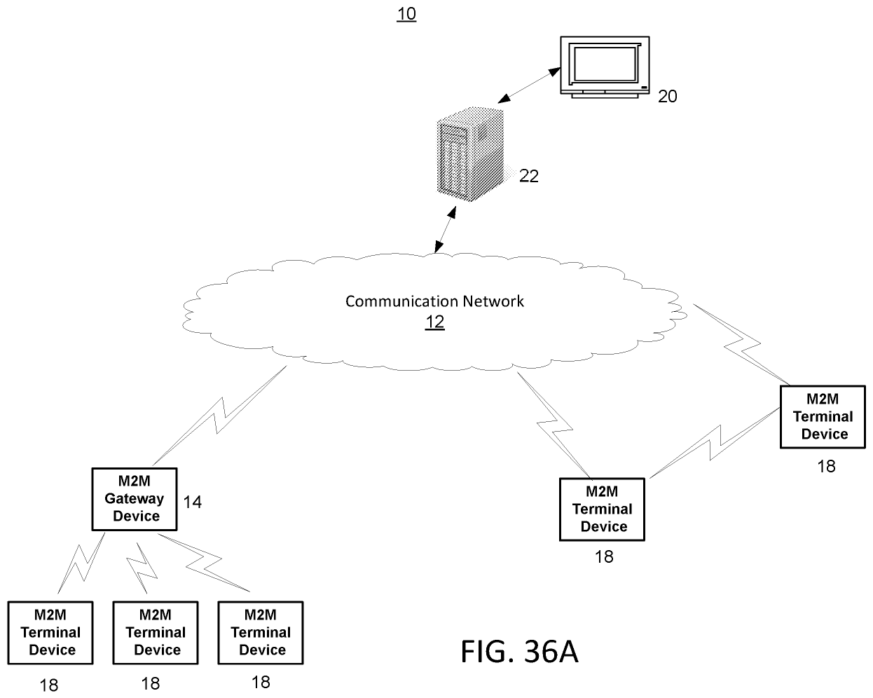

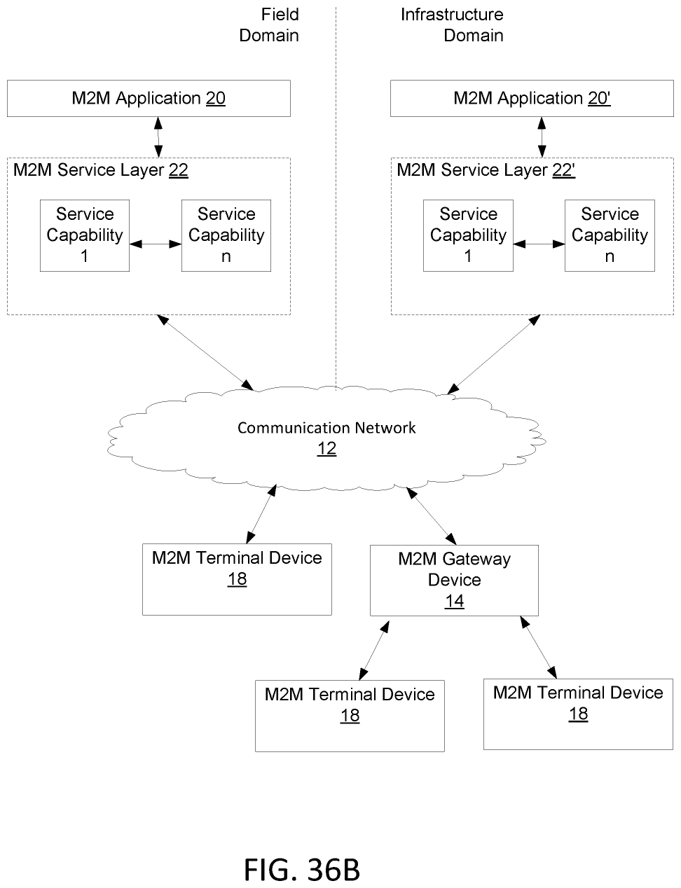

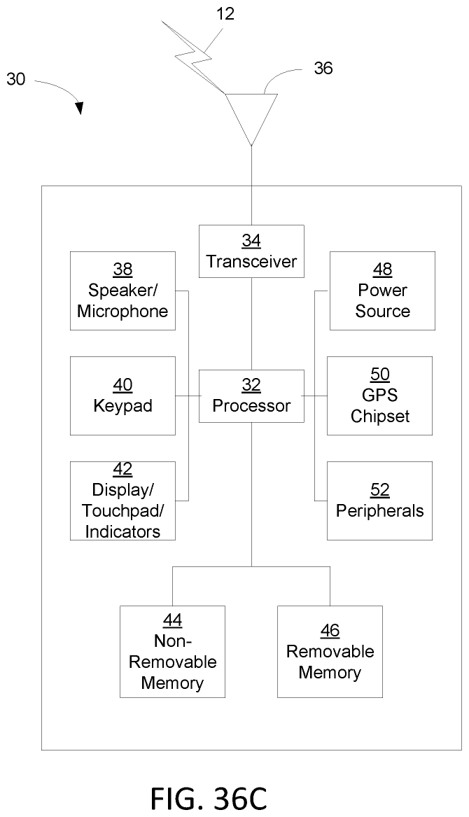

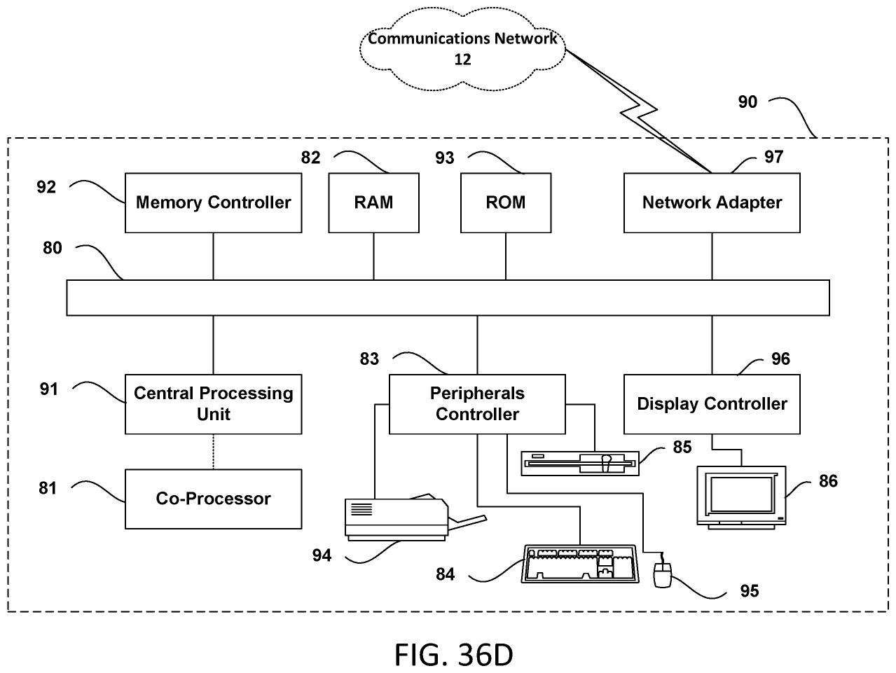

It is understood that the entities performing the steps illustrated herein, such as FIG. 5-7, FIG. 9-12, FIG. 17, FIG. 21, FIG. 22, FIG. 25, FIG. 27, or FIG. 29-34, may be logical entities. The steps may be stored in a memory of, and executing on a processor of, a device, server, or computer system such as those illustrated in FIG. 36C or FIG. 36D. In an example, with further detail below with regard to the interaction of M2M devices, SMS requestor 120 of FIG. 5 or IN-AE 250 of FIG. 33 may reside on M2M terminal device 18 of FIG. 36A, while SMS host 122, MN-CSE 253 or IN-CSE 251 of FIG. 33 may reside on M2M gateway device 14 of FIG. 36A. Skipping steps, combining steps, or adding steps between exemplary methods disclosed herein (e.g., FIG. 5, 11, 12, 17, 27, 29, 30, or 33) is contemplated.

FIG. 5 illustrates an exemplary overall procedure of SMS. At step 131, the SMS capability indication process when SMS Hosts register to each other. At this step 131, different SMS Hosts find what SMS capabilities other SMS Hosts can provide. At step 132, there is the Hierarchical SMJP association and generation process that may be triggered by an SMS Requestor 120 to enable the SMS Hosts to build Parent-Child relationships among different SMJPs distributed on them. This process may also be automatically triggered by a SMS Host. At step 133, after building the SMJP association, SMS Requestors 120 may send SMS requests to SMS Host 121 (Master SMS Host) for SMI creation based on the Hierarchical SMJP built in step 132. At step 134, is the process for creating Hierarchical SMI among SMS Host 121, SMS Host 122 and SMS Host 123 is disclosed based on the Hierarchical SMJP. This step may involve SMJP/SMI discovery. At step 135, SMS Host 121 may return the SMI creation result to the SMS Requestor 120 where the SMS request has previously issued to SMS Host 121. Steps 131-step 135 are disclosed in more detail herein.

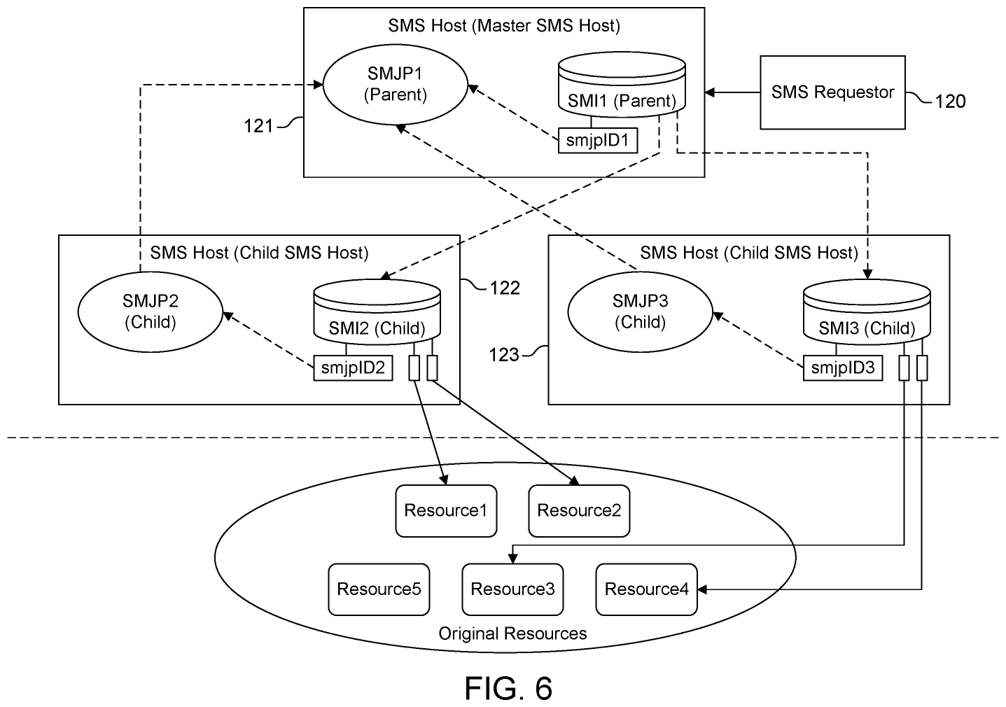

As shown in FIG. 6, the hierarchical relationships are illustrated among SMJPs and among SMIs on different SMS Hosts. SMJP1 on SMS Host 121 can be linked with SMJP2 on SMS Host 122, as well as SMJP3 on SMS Host 123. How to build the relationship among them is disclosed in more detail herein. Each SMI needs an SMJP since the SMJP may include the information that may be required for creating this SMI. The types of SMI in the network may be a parent SMI or child SMI. The child SMIs (e.g. SMI2 and SMI3) may be used as member or child resources of the parent SMI (e.g. SMI1). SMI1 may be created based on SMJP1 on SMS Host 121, SMI2 may be created based on SMJP2 on SMS Host 122 and SMI3 is created based on SMJP3 on SMS Host 123. Although FIG. 6 only shows an example of the Hierarchical SMI with two levels, a Hierarchical SMI with more than two levels are possible as well. For example, SMI2 may have other SMIs as its member or child resources, and then SMI1 is a three-level Hierarchical SMI.

Considering the use cases, when Server 106 as SMS Host 121 in Clinic A receives a request from a gateway 105 as the SMS Requestor, it leverages Server 107 as SMS Host 122 in Hospital B to mash up data resources from the private data on Server 107 or open data sources on public Server-M, and Server 108 as the SMS Host 123 in Hospital C to mash up data sources from the private data on Server 108 and open data on public Server-N, respectively. After that, SMS Host 121 may be able to generate a Hierarchical SMI based on the SMIs generated by SMS Host 122 and SMS Host 123.

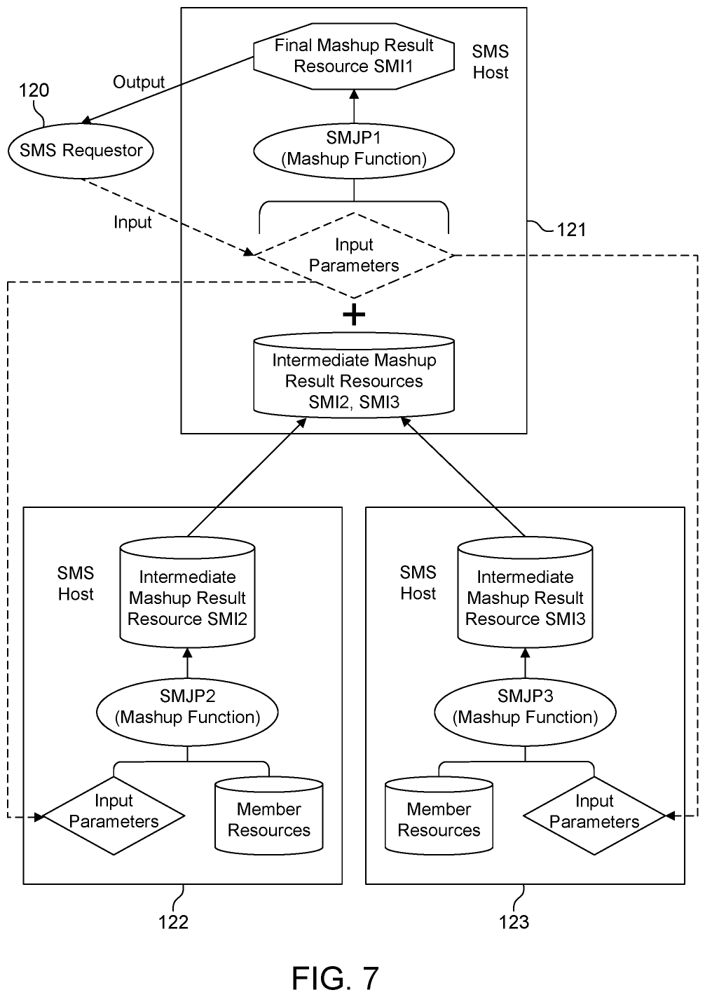

More specifically, FIG. 7 describes the functional operations of distributed semantic mashup. Upon receiving an SMS request from SMS Requestor 120 with or without input parameters for SMJP1, SMS Host 121 may send the corresponding request to SMS Host 122 for SMJP2 and to SMS Host 123 for SMJP3, with or without input parameters depending on SMJP2's and SMJP3′ configurations. Then, SMS Host 122 and SMS Host 123 create SMI2 and SMI3 with Intermediate Mashup Results, respectively. The SMI1 with Final Mashup Result may be created based on SMJP1 with Intermediate Mashup Results from SMI2 and SMI3, as well as the input parameters provided by SMS Requestor 120, if any. Finally, SMS Host 121 may return the Final Mashup Result to the SMS Requestor 120.

As disclosed in more detail herein, the SMS capability indication concept and procedure are disclosed for indicating SMS capabilities among SMS Hosts. The SMS discovery process and SMS capability update notification process are also disclosed.

Based on the SMS indication or SMS discovery, the process to generate a hierarchical SMJP is disclosed by correlating or connecting SMJPs from different SMS Hosts. This process may be triggered by SMS Requestor 120, or alternatively by SMS Hosts themselves to formulate hierarchical SMS and expose it to SMS Requestors.

The procedures of distributed semantic mashup for Hierarchical SMI generation with sequential execution are disclosed based on the dependency among different child SMJPs.

The procedures of distributed semantic mashup for Hierarchical SMI generation with parallel execution are disclosed with the consideration that there are no dependencies among child SMJPs.

The procedures of distributed semantic mashup for Hierarchical SMI generation without SMJP association are disclosed with the consideration that there is no SMJP association involved in the mashup process.

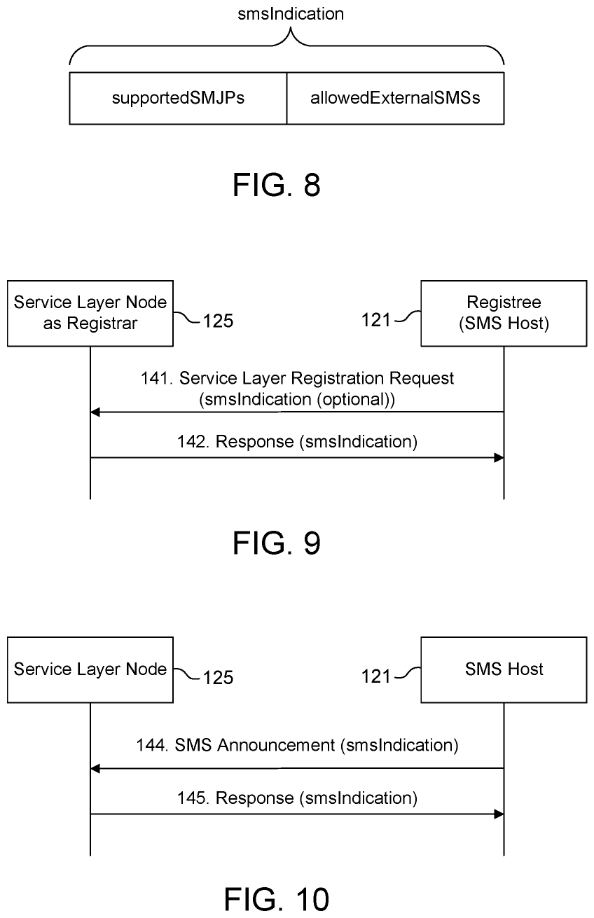

SMS Capability Indication and SMS Discovery—From the semantic mashup perspective, the types of entities in the service layer may include SMS Requestors, SMS Hosts, or Resource Hosts. The SMS Hosts have the capability to provide SMS to SMS Requesters while Resource Hosts hosting original resources may not provide SMS. An SMS Host may have an SMJP library, which includes SMJPs that may be run by some specific SMS(s) as hosted by this SMS Host. An SMJP may include attributes or child resources such as smjpID, semanticDescriptor, inputDescriptor, outputDescriptor, memberFilter and functionDescriptor. Before discovering the SMJPs, SMS Hosts may first be identified or discovered. The SMS indication and exchange may be used for this purpose. The SMS indication and exchange may be implemented during the service layer registration process or announcement process by sending or receiving messages with smsIndication parameter. As shown in FIG. 8, the smsIndication parameter in a registration message may include the following information:

The parameter supportedSMJPs includes a list of supported SMJPs' information. For each SMJP, its smjpID and semanticDescriptor (which describe their SMS capabilities) may be included in the list. This parameter indicates the SMSs supported by the node (e.g., SMS Host) which sends the smsIndication. For example, a single SMJP may indicate a SMS, a set of SMJPs together may indicate a SMS, or a SMJP may be used in multiple different SMSs.

There is another parameter called allowedExternalSMSs to indicate if external SMJPs may be used to create SMIs on the SMS Host which issued this smsIndication. Its value may be simply 1 which means this SMS Host may execute external SMS that may be hosted on other SMS Hosts; or 0 means this SMS cannot execute external SMS.

SMS Capability Indication Process—As shown in FIG. 9, if a Registree (e.g., SMS Host) registers to a Service Layer Node 125, they may indicate SMS capability to each other by including the smsIndication parameter in both the registration request and the registration response. At step 141, the Registree (e.g., SMS host 121) sends the Service Layer Node 125 a service layer registration request with smsIndication parameter to indicate its SMS capability if the Registree is an SMS host. If the Registree is an SMS Requestor (e.g., SMS requestor 120) or a Resource Host that has no SMS capability, then the smsIndication parameter may not be included in the registration request message. At step 142, Service Layer Node 125 receives smsIndication from the Registree and maintains it locally for the Registree, which allows other SMS Requestors or SMS Hosts to discover the Registree's smsIndication from the Server Layer Node 125. Then, the Service Layer Node 125 may send the response to the Registree, which may include a smsIndication parameter to indicate its SMS capabilities if it is a SMS Host as well.

Furthermore, an SMS Host 121 may actively send an SMS announcement with smsIndication parameter to other Service Layer Nodes to indicate its SMS capability in case other service layer nodes might be interested in its SMS, as shown in FIG. 10. At step 144, SMS Host 121 sends an SMS announcement to a Service Layer Node 125. The SMS announcement message may include smsIndication parameter to indicate its SMS capability. The Service Layer Node 125 receives smsIndication from the SMS Host 121 and maintains it locally, which allows other SMS Requestors or SMS Hosts to discover this SMS Host's smsIndication from the Server Layer Node 125. Then, it sends a response to the SMS Host 121; the response may also include smsIndicaton parameter to the SMS Host if the Service Layer Node 125 also supports SMS.

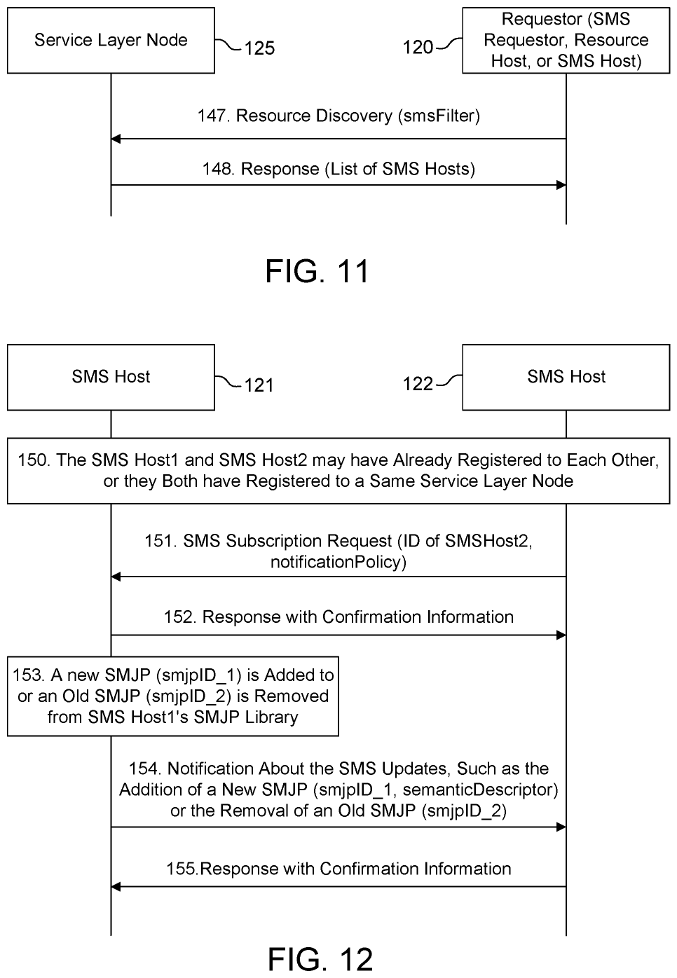

SMS Hosts Discovery Process—SMS Hosts may also be discovered through semantic discovery process by using smsFilter in the request message. By using the smsFilter, a list of SMS Hosts registered on the service layer node and their capabilities may be returned to the Requestor, which may be an SMS Requestor, an SMS Host, or a Resource Host. As shown in FIG. 11, at step 147, the Requestor 120 sends a resource discovery request to a Service Layer Node 125. The resource discovery request may include smsFilter, in which smsFilter may specify the preferred SMS with certain type of SMJPs that the requestor is interested in. At step 148, Service Layer Node 125 sends a response to the Requestor 120. This response message includes a list of URIs of the qualified SMS Hosts which satisfy the criteria described in the smsFilter. This message may also include the smsIndication of each discovered SMS Host if it is available to Service Layer Node 125.

In the disclosed DSMS, two or more SMS hosts work together to provide the mashup services. If one SMS Host has new SMS capabilities (e.g., due to an upgrade of the SMS Host, or the locally stored SMJPs have been changed), it may notify other SMS Hosts about this update so that they may utilize the new SMS capability. The procedure for this case is shown in FIG. 12. At step 150, SMS Host 121 and SMS Host 122 may have registered with each other or connected by a same service layer node. At step 151, SMS Host 122 may send an SMS subscription request with SMS Host 122's ID, notificationPolicy to SMS Host 121 in order to get notifications when the SMS Host 121 has updated SMS capabilities. At step 152, SMS Host 121 sends a response to confirm the subscription to SMS Host 122. At step 153, a new SMJP with ID smjpID_1 is added to (or an old SMJP with ID smjpID_2 is removed from) the SMS Host 121's SMJP library. At step 154, SMS Host 121 sends the notification about the addition of the new SMJP with smjpID_1 and its semanticDescriptor, which describes its SMS capability (or the removal information of an old SMJP with smjpID_2) to SMS Host 122. At step 155, SMS Host 122 may respond to confirm if it successfully receives the notification from SMS Host 121.

Hierarchical SMJP Association and Generation—SMS Hosts may indicate or exchange their SMS capabilities in the registration process. After an SMS Host receives an SMS indication from another SMS Host, it may conduct local discovery to see if there are any interesting SMJPs that may match and connect to their existing SMJPs in the SMJP library based on the semantic description of SMJPs, in order to build more useful and powerful hierarchical SMJPs. In fact, in the SMS indication process, it is possible that only semanticDescriptor of each SMJP is given. Thus, it is not sufficient to determine if two SMJPs may be matched and connected to build a hierarchical SMJP. So two complete SMJPs must be placed on one SMS Host so that they may be completely compared and matched. Alternatively, SMS Hosts may indicate and exchange more information (other than semanticDescriptor) about their SMJPs.

There are multiple scenarios to consider. In an exemplary scenario 1, SMS Host 121 with SMJP1 that finds an interesting SMJP2 from another SMS Host 122 may pull the complete SMJP2 from SMS Host 122 to itself to determine if SMJP1 and SMJP2 may be associated, and then notify the SMS Host 122 about the association relationship.

In an exemplary scenario 2, SMS Host 121 with SMJP1 that finds an interesting SMJP2 from another SMS Host 122 may push the complete SMJP1 to SMS Host 122 to ask it to determine if SMJP1 and SMJP2 may be associated, and then return the results to SMS Host 121.

In an exemplary scenario 3, SMS Host 121 just finds an interesting SMJP2 from SMS Host 122 and SMJP3 from SMS Host 123 based on their semanticDescriptors that may have the potential to be associated with a new SMJP on SMS Host 121. In this scenario, SMS Host 121 needs to pull SMJP2 and SMJP3 from SMS Host 122 and SMS Host 123, respectively. Then, SMS Host 121 may create a new SMJP based on SMJP2 and SMJP3 and notify both SMS Host 122 and SMS Host 123 about the association relationship.

After the association relationship is formed among two or more SMJPs, an approach is needed to model this relationship in each SMJP. Firstly, disclosed herein is an attribute called relatedSMJPs to model its relationship with other SMJPs. Secondly, disclosed herein are possible solutions for the scenario 1-scenario 3. Thirdly, a possible solution for hierarchical SMJP's modification and deletion is disclosed. Note that the SMJP association process may be triggered when SMS Hosts register to each other, or may be triggered by SMS Requestors when receiving the SMS requests. The specific trigger setup depends on the SMS Host's configurations.

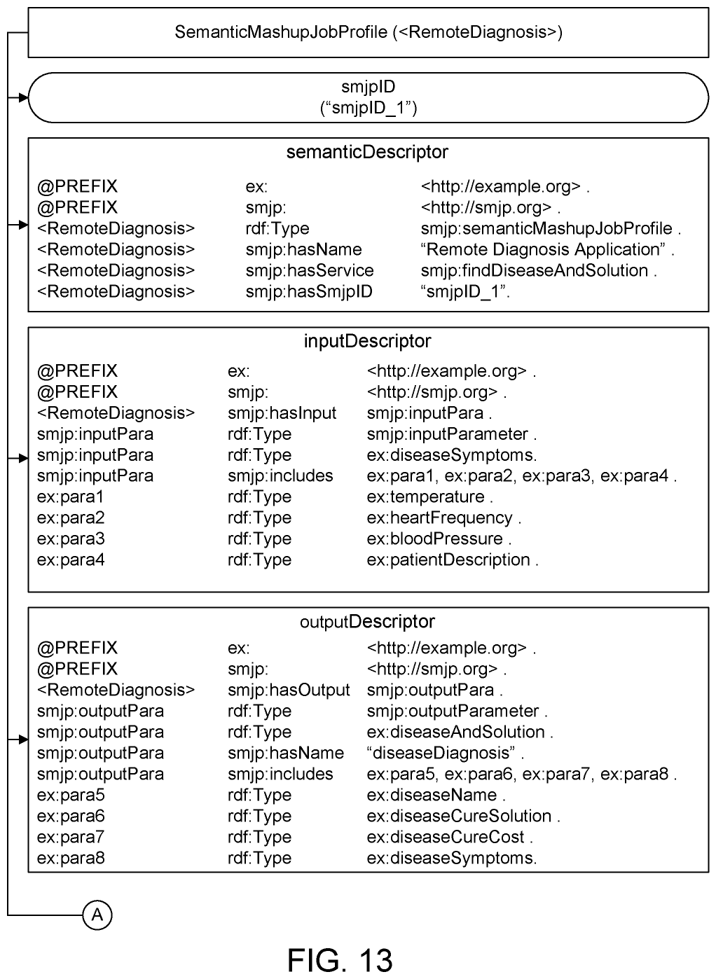

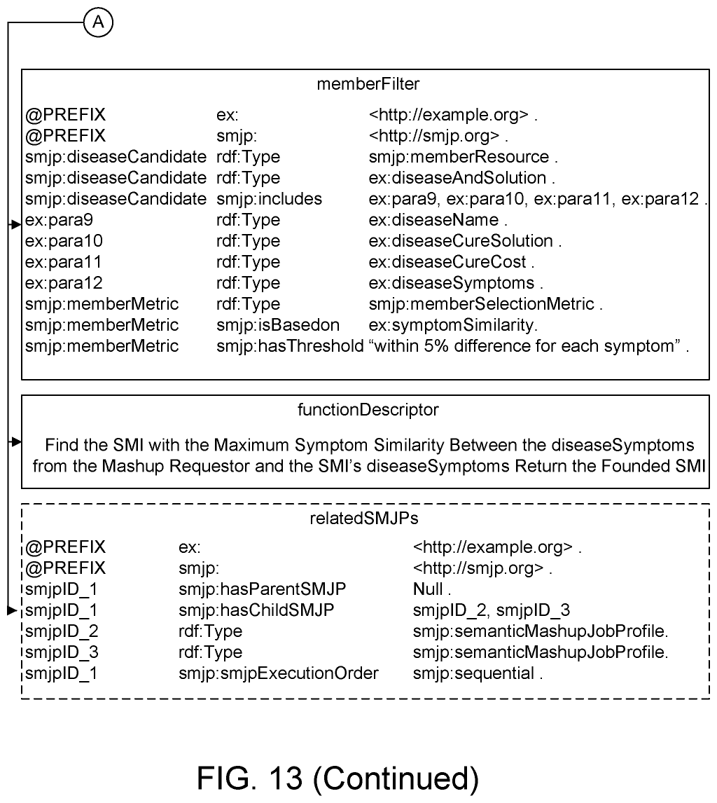

Hierarchical SMJP Modeling—As shown in the service layer semantic mashup service, a SMJP resource in an SMJP library may include attributes or child resources such as smjpID, semanticDescriptor, inputDescriptor, outputDescriptor, memberFilter and functionDescriptor. Considering the Smart eHealth application as an example, a sample SMJP representation for SMJP1 (“RemoteDiagnosis”) is shown in FIG. 13, where it is assumed that SMJP1 uses SMJP2 and SMJP3 as its child SMJP. In order to enable hierarchical SMJP association and generation, relatedSMJPs is introduced as a new attribute of an SMJP, which is detailed as follows.

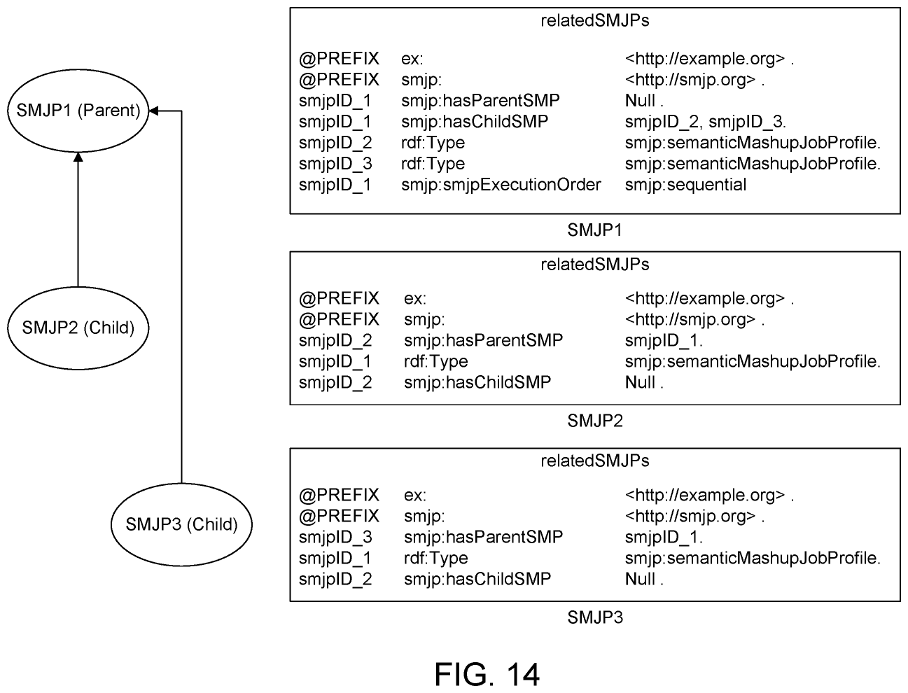

The relatedSMJPs may include a number of parent SMJPs and child SMJPs that might be needed during the hierarchical SMI generation process. The relatedSMJPs is described by metadata models, such as RDF triples. By obtaining the metadata of the relatedSMJPs, the SMS Host may understand where to send the child SMI discovery or SMI creation request and based on which Child SMJP, as well as in what order. One example is shown in FIG. 14 for Smart eHealth application, SMJP1 has no Parent SMJPs and has two Child SMJPs, which are SMJP2 located on SMS Host 122 and SMJP3 located on SMS Host 123, respectively. Note that SMS Host 121 may discover SMJP2 and SMJP3 using the methods described in FIG. 9-FIG. 12, for example. For the SMJP2 and SMJP3's relatedSMJPs, both of them may have the same Parent SMJP which is SMJP1 and have no Child SMJPs. Specifically, the triple “smjpID_1 SMJP:hasChildSMJP smjpID_2, smjpID_3” means SMJP1 with smjpID_1 has two Child SMJPs whose IDs are smjpID_2 and smjpID_3, respectively. The triple “smjpID_1 SMJP:SMJPExecutionOrder SMJP:sequential” means SMJP1 with smjpID_1 may execute its Child SMJPs in sequential order. Based on the SMJP order appearing in this triple “smjpID_1 smjp:hasChildSMJP smjpID_2, smjpID_3”, the SMJP2 with smjpID_2 must be executed first and then SMJP3 with smjpID_3 may be executed when SMJP2's execution is completed. Alternatively, a separate triple such as “smjpID_1 smjp:childSMJPExecutionOrder smjpID_2, smjpID_3” may be used to explicitly indicate the execution order of child SMJP. If triple is “smjpID_1 SMJP:SMJPExecutionOrder SMJP:parallel”, then SMJP2 and SMJP3 may be executed in parallel.

The term smjp:memberMetric may be used to define the evaluation criteria regarding to how to select member resources for a given SMJP (as a parent SMJP) and those member resources may also be SMJPs (as a child SMJPs). For an example, a metric criterion may be “simple match” in the sense that the outputs of the child SMJPs match the criteria as specified in the memberFilter of the parent SMJP. For example, a potential disease provided by a child SMJP may be a member resource of the parent SMJP if the symptoms as described in the output of the child SMJP are the same as the symptoms as specified by the SMS Requestor of the parent SMJP (this is a qualitative approach). Note that, during the matching process, it may be possible that the symptoms (as specified by the SMS Requestor) or the diseases (as diagnosed by Child SMS Hosts) are described using an ontology A while the symptoms/diseases included by the child SMJP are described by a different ontology B. In such a case, certain ontology mapping and alignment should be leveraged in order to decide whether the two or more different symptoms or diseases are the same. In a more advanced scenario, the member metric may be more complicated. For example, it is not only required that the symptoms included in the output of the child SMJP are as the same as the symptoms specified by the SMS Requestor of the parent SMJP, but also that the values of those symptoms should be close enough. The reason is that in many cases, a quantitative approach beyond the qualitative approach may be more appropriate. For example, although two patients may both have the fever symptom, they may have different diseases because one patient has a low fever while the other has a high fever. As shown in the smart eHealth example, it is defined that the member selection criteria may be based on the “symptom similarity”. In other words, for a given set of disease symptoms as provided by the SMS Requestor (as described by the inputDescriptor), only the member resources that have the similar disease symptoms may be selected as the member resources for this parent SMJP. The similarity threshold definition is that for each of the symptoms, the difference between the value provided by the SMS Requestor and the value provided by the potential member resource candidate should be less than 5% difference.

Hierarchical SMJP Association Process —The association between two SMJPs residing on different SMS Hosts depends on their semantic modeling's matching relationship. The conditions that may be met in order to build Parent-Child relationship between two SMJPs (e.g. SMJP1 and SMJP2) are listed as follows. In a first example, the output parameters of child SMJP (e.g., SMJP2) satisfy the member filter of parent SMJP (e.g., SMJP1); and a second example, the input parameters of child SMJP (e.g., SMJP2) may also be provided by parent SMJP (e.g., SMJP1), which may be confirmed by investigating the content of inputDescriptor of both child and parent SMJPs (e.g., SMJP2 and SMJP1).

Note that if input parameters that are provided by SMS Requestor are only used by the parent SMJP, the following conditions may be used to build parent-child association relationship: the output parameters of the child SMJP satisfy the member filter of parent SMJP.

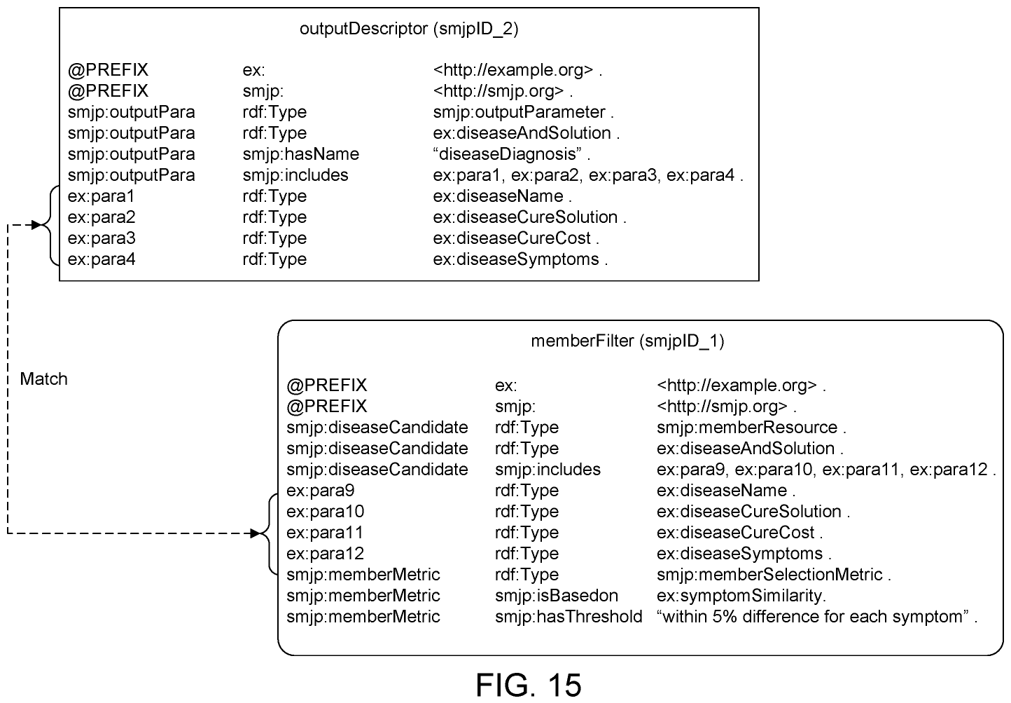

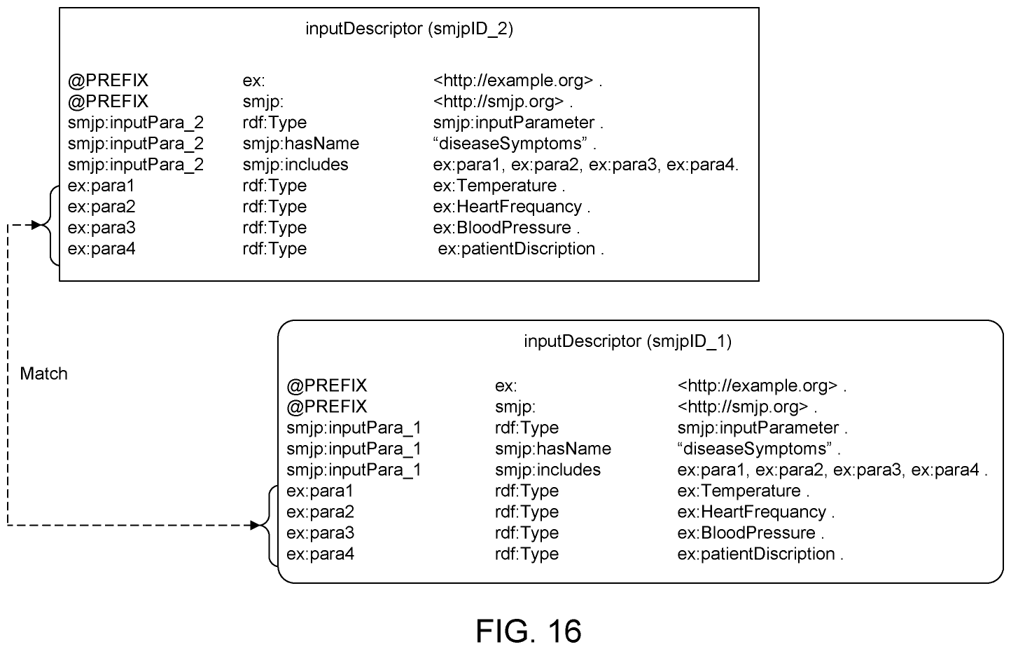

Then, SMJP1 may be considered as the Parent SMJP of SMJP2 and SMJP2 may be considered as the Child SMJP of SMJP1. Normally, the SMJP from the SMS Host that directly received SMS request may be the Hierarchical SMJP. For example, in the Smart eHealth application shown in FIG. 15, the output parameter descriptions of outputDescriptor of SMJP2 fully match the mashup member descriptions of memberFilter of SMJP1. In addition, as shown in FIG. 16, the input parameters of SMJP2 may also be provided by the input parameters of SMJP1 by matching their descriptions. By the way, the number of input parameters in SMJP2 may be less than the number of input parameters in SMJP1 but the set of input parameters of SMJP2 may be provided by the set of input parameters of SMJP1. If all of them hold true, then SMJP1 may be associated with SMJP2 as a Parent-Child relationship, which means the SMI that is generated based on Child SMJP2 may be used as a member resource of SMI generated based on Parent SMJP1.

An example below may be used to illustrate how to build a Hierarchical SMJP. Assume two SMS Hosts, SMS Host 121 and SMS Host 122, are registered to each other. SMJP1 is an SMJP in the SMJP library located on SMS Host 121, while SMJP2 is an SMJP in the SMJP library located on SMS Host 122. A purpose may be to decide if it is possible to associate SMJP1 with SMJP2 to build Parent-Child relationship and then create the Parent-Child relationship if the association conditions are met. Exemplary approaches are disclosed below.

In a first approach, an idea is to let SMS Host 121 to pull the complete SMJP2 from SMS Host 122 to determine if SMJP1 and SMJP2 may be associated, and then notify SMS Host 122 about the association relationship. As shown in FIG. 17, the procedure for building potential parent-child association between SMJP1 and SMJP2 on the master SMS Host, which is SMS Host 121 in this example, may be as follows. At step 160, SMS Host 121 may have already analyzed the metadata of SMJP1 and figured out which types of SMJPs may be its child SMJP. Using SMS indication or discovery procedures as described herein, SMS Host 121 may find that SMJP2 with smjpID_2 from SMS Host 122 is a potential child for SMJP1 based on their semanticDescriptors, which describe the capabilities of the SMJPs. In order to verify if this is the case, SMS Host 121 may retrieve more information of SMJP2 from SMS Host 122. At step 161, SMS Host 121 may send an SMJP pull request to SMS Host 122. The parameter in this request may include the following:
smjpID_2: the URI of SMJP2 on SMS Host 122. The value of this parameter has been obtained via Step 160.

At step 162, after receiving the SMJP pull request from SMS Host 121 in step 161, SMS Host 122 may check if SMS Host 121 has the permission to access SMJP2's representation which may include the information of SMJP2. If yes, SMS Host 122 may respond with SMJP2's representation; otherwise, SMS Host 122 may deny the request. At step 163, SMS Host 122 may send the response with SMJP2's representation to SMS Host 121, which may include inputDescriptor, outputDescriptor, functionDescriptor, memberFilter, or semanticDescriptors. At step 164, based on SMJP2's representation received from step 163, SMS Host 121 may determine if the output parameters described in SMJP2's outputDescriptor match the member resources criteria described in SMJP1's memberFilter; SMS Host 121 may determine if the mashup metrics in memberFilter of SMJP1 and SMJP2 match; SMS Host 121, in addition, may determine if the input parameters in inputDescriptor of SMJP2 (if exists) may be provided by the input parameters in inputDescriptor of SMJP1, as the whole set or a subset.

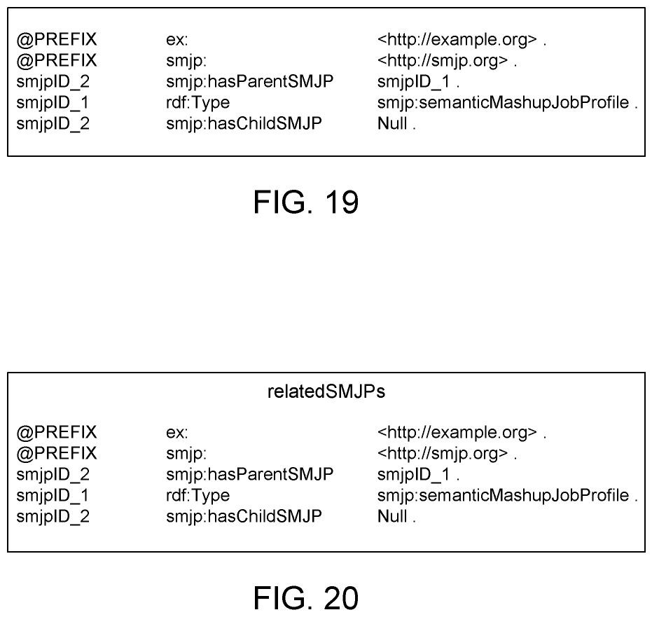

With continued reference FIG. 17, at step 165, if all matches (e.g., determinations) in step 164, SMS Host 121 links SMJP2 with SMJP1 in the SMJP1's relatedSMJPs attribute by adding related semantic descriptions. For instance, the relatedSMJPs in SMJP1 may be modified as shown in FIG. 18. At step 166, SMS Host 121 may send a request to SMS Host 122 to build the parent-child relationship association between SMJP1 and SMJP2. As shown in FIG. 19, the parameter in the message is using RDF Triples which indicates SMJP1 is the parent SMJP of SMJP2. At step 167, upon receiving the request with RDF Triples from step 166, SMS Host 122 may take those RDF triples included in step 166 and insert them into SMJP2's relatedSMJPs to reflect the parent-child relationship between SMJP1 and SMJP2. For instance, the relatedSMJPs in SMJP2 may be modified as shown in FIG. 20. At step 168, SMS Host 122 may respond to SMS Host 121 to confirm if the parent-child association relationship between SMJP1 and SMJP2 has been successfully created in SMJP2.

In a second approach, the idea is to let SMS Host 121 push the complete SMJP1 to SMS Host 122 to let SMS Host 122 determine if SMJP1 and SMJP2 may be associated, and then return the association relationship to SMS Host 121. As shown in FIG. 21, the procedure for building potential parent-child association between SMJP1 and SMJP2, where the procedures for checking and comparing relevant SMJPs is done on child SMS Host side which is SMS Host 122 in this example, and the association relationship is returned to SMS Host 121 are as follows. At step 170, SMS Host 121 has already analyzed the metadata of SMJP1 and figured out which types of SMJPs may be its child SMJP. Using SMS indication or discovery as described herein, SMS Host 121 finds an SMJP2 with smjpID_2 from SMS Host 122 is a potential child for SMJP1 based on their semanticDescriptors, which describes the capabilities of the SMJPs. In order to verify if this is the case, more information may be needed to confirm the relationship between SMJP1 and SMJP2. At step 171, SMS Host 121 sends an SMJP association request with SMJP1's representation, and SMJP2′ ID which is smjpID_2 to the SMS Host 122. At step 172, after receiving the association request from SMS Host 121 in step 171, SMS Host 122 checks if the output parameters in SMJP2's outputDescriptor match the parameters in SMJP1's memberFilter; SMS Host 122 also checks if the mashup metrics in memberFilter of SMJP1 and SMJP2 are matched; check if the input parameters in SMJP2's inputDescriptor (if exists) may be provided by the input parameters in SMJP1's inputDescriptor.

With continued reference to FIG. 21, at step 173, if all matches in step 172, SMS Host 122 creates the RDF Triples in SMJP2's relatedSMJPs to link SMJP2 with SMJP1, otherwise return denial information. For instance, the relatedSMJPs in SMJP2 may be modified as shown in FIG. 20. At step 174, SMS Host 122 responds to SMS Host 121 to confirm the SMJP association between SMJP1 and SMJP2. At step 175, SMS Host 121 may create RDF Triples in SMJP1's relatedSMJPs to associate SMJP1 with SMJP2 for the parent-child relationship. For instance, the relatedSMJPs in SMJP1 may be modified as shown in FIG. 18.

Furthermore, a service layer node or an SMS host may retrieve existing SMJPs from other SMS Hosts to create new SMJPs. The idea is to let an SMS Host search and retrieve some interesting SMJPs that may be combined in a logical way. For example, an SMS Host may combine a fever diagnosis SMJP and an Ebola diagnosis SMJP to provide a combined service for diagnosis of both diseases for an SMS Requestor. This new SMJP may be defined as an integration of two or more child SMJPs and may provide advanced and integrated SMS that combines the SMSs provided by the child SMJPs. In this way, the SMS capability of this SMS Host may be more powerful and useful. As shown in FIG. 22, the procedure is as follows. At step 180, using SMS indication or discovery, SMS Host 121 may discover an interesting SMJP2 with smjpID_2 from SMS Host 122, and an interesting SMJP3 with smjpID_3 from SMS Host 123, based on their semanticDescriptors which describe their capabilities. At step 181, SMS Host 121 sends an SMJP pull request with smjpID_2 to SMS Host 122. SMS Host 121 also sends an SMJP pull request with smjpID_3 to SMS Host 123. At step 182, after receiving the pull request from SMS Host 121, SMS Host 122 may determine if SMS Host 121 has the permission to access SMJP2's representation that includes the semantic information of SMJP2 associated with smjpID_2. If yes, SMS Host 122 responds with SMJP2's representation to SMS Host 121, otherwise, it may deny the request from SMS Host 121. Similarly, SMS Host 123 also determines if SMS Host 121 has the permission to access SMJP3's representation associated with smjpID_3 that includes the semantic information of SMJP3. If yes, SMS Host 123 may respond with SMJP3's representation to SMS Host 121, otherwise, it may deny the request from SMS Host 121. At step 183, based on the checking process in step 182, SMS Host 122 responds with SMJP2's representation to SMS Host 121. SMS Host 123 also responds with SMJP3's representation to SMS Host 121.

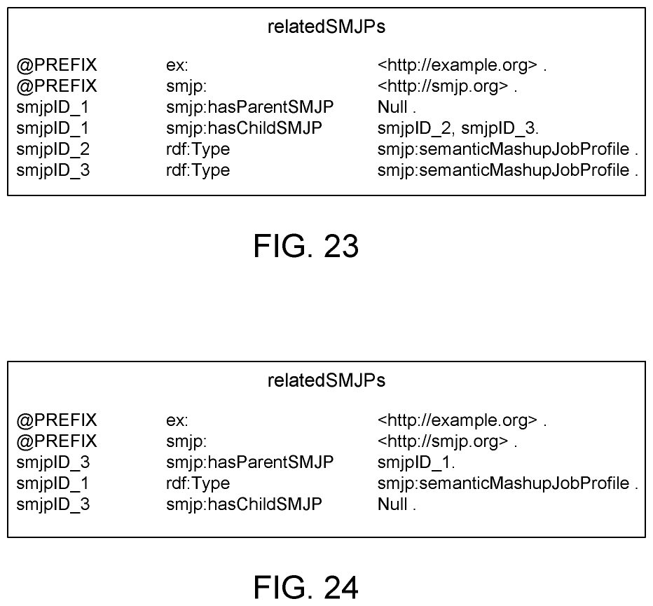

With continued reference FIG. 22, at step 184, after obtaining the representations of SMJP2 and SMJP3 from step 183, SMS Host 121 may generate a new SMJP with ID smjpID_1 by combining the representations of SMJP2 and SMJP3 together to provide a combined mashup service. SMS Host 121 may create the RDF Triple in SMJP1's relatedSMJPs to associate SMJP1 with SMJP2 and SMJP3. The relatedSMJPs in SMJP1 is shown in FIG. 23. At step 185, after creating SMJP1 in step 184, SMS Host 121 may send an SMJP association request with smjpID_1 and its semanticDescriptor, as well as SMJP2′ ID smjpID_2 to SMS Host 122. Similarly, SMS Host 121 also sends an SMJP association request with smjpID_1 and its semanticDescriptor, as well as SMJP3′ ID smjpID_3 to SMS Host 123. At step 186, after receiving the association request from SMS Host 121, SMS Host 122 may create RDF Triples in SMJP2′ relatedSMJPs to link SMJP2 with SMJP1 for the parent-child relationship. For instance, the relatedSMJPs in SMJP2 may be modified as shown in FIG. 20. Similarly, SMS Host 123 may create RDF statement in SMJP3′ <relatedSMJPs> to link SMJP3 with SMJP1 for the parent-child relationship. For instance, the <relatedSMJPs> in SMJP3 may be modified as shown in FIG. 24. At step 187, SMS Host 122 may respond to SMS Host 121 that the parent-child association relationship between SMJP1 and SMJP2 has been successfully created in SMJP2 or not. Similarly, SMS Host 123 also responds to SMS Host 121 that the parent-child relationship between SMJP1 and SMJP3 has been successfully created in SMJP3 or not. At step 188, if all the responses are error code, then SMS Host 121 delete this SMJP with smjpID_1; if some of responses are error code (e.g., response from SMS Host 123 is error code), then modify the SMJP1 accordingly by removing SMJP3's triples from SMJP1.

Hierarchical SMJP Modification and Deletion Process—The relatedSMJPs resource may be used for modeling the relationship between SMJPs on the same or different SMS Hosts. It is possible that an SMJP may be changed by the belonging SMS Host. Considering the Smart eHealth use case herein, if Server 108 in Hospital C removes SMJP3 from its SMJP library which may mean Hospital C cannot continually serve this type of diagnosis request online and may only serve the diagnosis request offline. In this scenario, Server 106 in Clinic A may remove SMJP3 from the relatedSMJPs of SMJP1 since Clinic A cannot send the request to Server 108 for SMJP3 when handling the SMS request based on SMJP1. The solution may leverage the SMS Hosts Capability Update Notification Process disclosed herein. As shown in FIG. 25, the procedure for hierarchical SMJP modification and deletion is as follows. At step 190, SMS Host 121 and SMS Host 122 may have already registered to each other, and subscribed on each other's SMS capability update notification. SMS Host 121 may have an SMJP1 with smjpID_1 that may have a parent-child relationship with SMJP2 with smjpID_2 on SMS Host 122. Assume that SMJP2 is deleted by SMS Host 122. At step 191, due to the update of SMS Host 122′ SMS capabilities, SMS Host 122 mays send a notification to SMS Host 121 to inform it that SMJP2 as denoted by smjpID_2 has been deleted. At step 192, after receiving the SMS notification from SMS Host 122 in step 191, SMS Host 121 may check the SMJPs that have relatedSMJPs attribute. SMS Host 121 may find that the SMJP1 with smjpID_1 has the parent-child relationship with SMJP2. The original relatedSMJPs in SMJP1 may be as shown in FIG. 23. At step 193, since SMJP1 is identified to have a parent-child relationship with SMJP2 in step 192, SMS Host 121 may modify its relatedSMJPs and may delete the triples that describes the relationship between SMJP1 and SMJP2. For instance, the relatedSMJPs in SMJP1 may be modified as shown in FIG. 26. At step 194, after successfully modifying SMJP1's relatedSMJPs attribute and deleting the triples describing the relationship between SMJP1 and SMJP2, SMS Host 121 may respond with confirmation information to SMS Host 122.

Distributed Semantic Mashup for Hierarchical SMI Generation with Sequential Execution Dependency—Considering the Smart eHealth use case disclosed herein, Server 106 in Clinic A may first send the diagnosis request to Hospital B, and only send the request to Hospital C when the diagnosis result from Hospital B is really something serious or a special disease that needs to be confirmed with the result from Hospital C, since Hospital C may only perform the diagnosis depending on the diagnosis result from Hospital B. For example, Hospital B may be a general hospital (e.g., a hospital that is used to treating relatively common ailments) while Hospital C may be a more specialized hospital (e.g., a hospital that is used to treating relatively uncommon ailments or particularly life threatening). In this scenario, the sequential execution dependency of various SMJPs among each other may be significant in generating the intended results. Using the approaches herein (e.g., first approach or second approach), hierarchical and cascaded association relationship may be created among multiple SMJPs. For example, SMJP1 is the Parent SMJP of SMJP2, while SMJP2 is the Parent SMJP of SMJP3. But it is also possible to create a hierarchical association relationship where the parent SMJP has multiple child SMJPs; furthermore, those multiple child SMJPs may have certain dependencies or they may have to be executed in a certain sequence in order to calculate the right mashup result. This issue may be about how to create Semantic Mashup Instance (SMI) after establishing hierarchical SMJP using the procedures herein, for example. This issue is addressed herein.

In the Smart eHealth use case, SMS Host 121 has an SMJP1 that depends on Child SMJPs in SMS Host 2 and SMS Host 123. The SMS Requestor 120 has already discovered the SMJP with smjpID_1 from SMS Host 121. As shown in FIG. 13, the relatedSMJPs attribute of smjpID_1 indicates that the execution of SMJP3 is dependent on the execution completion of SMJP2. This forms a sequential execution process based on the dependency between SMJP2 and SMJP3. As shown in FIG. 27, the procedure for creating distributed mashup instances with sequential execution dependency may be as follows. At step 200, the SMJP association relationships between SMJP1 with smjpID_1 and SMJP2 with smjpID_2, SMJP1 with smjpID_1 and SMJP3 with smjpID_3 may have already been built between SMS Host 121 and SMS Host 122, SMS Host 121 and SMS Host 123. SMS Requestor 120 (e.g. User) may have already discovered SMJP1 with smjpID_1. At step 201, SMS Requestor 120 may send an SMI creation request to SMS Host 121. The SMI creation request may include the ID of the applied SMJP (e.g., the value of smjpID), as well as the input parameters defined by its inputDescriptor.

At step 202, after receiving the creation request, SMS Host 121 may check if the inputs smjpID_1 and inputPara from the SMS Requestor 120 are valid. If they are invalid, it may return an error code; if they are valid, it may check SMJP1's relatedSMJPs attribute and finds that SMJP1 has SMJP associations with SMJP2 on SMS Host 122 and SMJP3 on SMS Host 123. Also, there may be a dependency between SMJP2 and SMJP3 (e.g., the execution of SMJP3 depends on the execution completion of SMJP2 in this example). According to this dependency, SMS Host 121 may know (e.g., have determined or otherwise obtained) that the intermediate mashup result from SMS Host 122 based on SMJP2 should be calculated first and before the intermediate mashup result from SMS Host 123 based on SMJP3. Then, SMS Host 121 may calculate the final mashup result based on the intermediate mashup results from SMS Host 122 and SMS Host 123, respectively. However, if the first intermediate mashup result from SMS Host 122 cannot be obtained or is unavailable, there should be no need to calculate the intermediate mashup result based on SMJP3 due to the dependency between SMJP2 and SMJP3. At step 203, SMS Host 121 may send an SMI discovery or creation request with parameters smjpID_2, inputPara, maintenancePolicy to SMS Host 122. The parameters in the message sent to SMS Host 122 may be as follows:
smjpID_2: the URI of SMJP2 on SMS Host 122;
inputPara1: the value of input parameters that are defined and described by the inputDescriptor resource in the applied SMJP2 with smjpID_2;
maintenancePolicy1: the maintenance policy about how to report to the parent SMS Host 121 about the future changes about the corresponding SMI that may be discovered/created, e.g. if the child SMI is deleted or updated, the Child SMS Host 122 may report this to the Parent SMS Host 121, etc.

At step 204, upon receiving the request from SMS Host 121, SMS Host 122 may discover if there is an existing SMI2 satisfying the given smjpID_2 and inputPara1. If yes, add the maintenance policy maintenancePolicy1 to this SMI2's maintenance policy list that may determine how the future changes of SMI2 may be reported to the master SMS Host 121, and go to step 206, otherwise go to step 205. Note that each SMI resource may have a maintenance policy list which stores policies related to how to maintain this SMI resource (e.g., any actions to be taken if one of mashup members of SMI resource becomes unavailable or unreachable). At step 205, based on the fact that if no existing SMI2 satisfying the given smjpID_2 and inputPara2 is found in step 204, SMS Host 122 may create an SMI2 based on those parameters given in Step 3 such as smjpID_2, inputPara2, and maintenancePolicy2. In order to fully create SMI2, SMS Host 122 may need to discover and determine mashup members of SMI2 based on the memberFilter information included in SMJP2, which is denoted by smjpID_2.
If SMI2 is successfully created, SMS Host 122 returns its URI which is the value of smiID_2 to SMS Host 122; otherwise respond with error information to SMS Host 121.

At step 206, SMS Host 122 may respond to SMS Host 121 with the URI of SMI2 which is the value of smiID_2 if the SMI2 has been successfully discovered or created; otherwise it may return error information to SMS Host 121. At step 207, SMS Host 121 determine if SMS Host 122 successfully discovered or created SMI2 and returned the URI which is smiID_2. If SMI2 was successfully discovered or created, go to step 208; otherwise, due to SMJP3's execution dependency on SMJP2 as described in the relatedSMJPs of SMJP1 with smjpID_1, the SMI discovery or creation request to SMS Host 123 and subsequent SMI3's generation may be cancelled, and go to step 214. At step 208, upon the creation of SMI2 based on SMJP2 in step 207, SMS Host 121 may send an SMI discovery or creation request with parameters smjpID_3, inputPara, and maintenancePolicy to SMS Host 123.
smjpID_3: the URI of SMJP3 on SMS Host 123;
inputPara2: the input parameters that are defined and described by the inputDescriptor resource in the applied SMJP3 with smjpID_3;
maintenancePolicy2: the maintenance policy about how to report to the Parent SMS Host 121 about future changes with the corresponding SMI that may be created.

At step 209, upon receiving the request from SMS Host 121, SMS Host 123 may discover if there is an existing SMI3 satisfying the given smjpID_3 and inputPara2. If yes, add the maintenance policy maintenancePolicy2 to this SMI3's maintenance policy list that may determine how future changes of SMI3 may be reported to the master SMS Host 121, and go to step 211; otherwise, go to step 210. At step 210, Based on the fact that no existing SMI3 satisfying the given smjpID_3 and inputPara is found in step 209, SMS Host 123 may create an SMI3 based on those parameters given in step 208 such as smjpID_3, inputPara2, and maintenancePolicy2. If SMI3 is successfully created, SMS Host 123 returns its URI that is the value of smiID_3; otherwise, SMS Host 123 responds with error information to the SMS Host 121. At step 211, SMS Host 123 responds to SMS Host 121 with URI of SMI3 that is the value of smiID_3 if the SMI3 has been successfully created or discovered; otherwise, SMS Host 123 returns error information to SMS Host 121.

At step 212, SMS Host 121 checks if the SMS Host 123 successfully created SMI3 and returned the URI which is smiID_3, and go to Step 213; otherwise, go to step 214. At step 213, based on the successful creation of SMI2 and SMI3, SMS Host 121 creates an SMI1 with smiID_1 based on: 1) member resources: SMI2 with smiID_2 and SMI3 with smiID_3; 2) input parameters: inputPara; and 3) the maintenancePolicy. If SMI1 is successfully created, SMS Host 121 returns the URI of SMI1 to SMS Requestor; otherwise, it returns an error code to SMS Requestor. At step 214, SMS Host 121 responds with the URI of SMI1 that is the value of smiID_1 to the SMS Requestor 120 if SMI1 is created; otherwise, SMS Host 121 returns error code to SMS Requestor 120 if the creation of SMI1 is failed. Alternatively, SMS Host 121 may be able to calculate the semantic mashup result and return it directly to SMS Requestor 120.

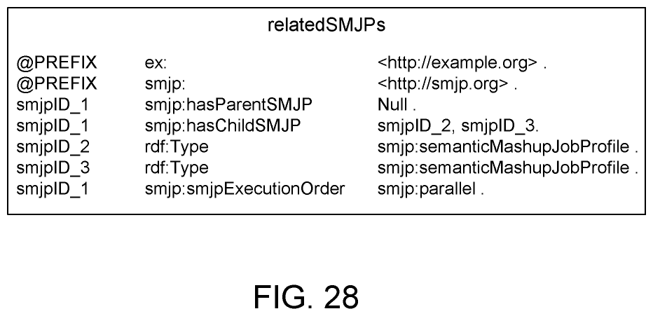

Distributed Semantic Mashup for Hierarchical SMI Generation with Parallel Execution without Dependency—Considering the Smart eHealth use case herein, Server 106 in Clinic A may send the diagnosis requests to Hospital B and Hospital C at the same time if the diagnosis results from the two hospitals have no direct impact on each other. For example, Hospital C's diagnosis results may not depend on the diagnosis results from Hospital B. In this scenario, the parallel execution is appropriate in generating the results. More specifically, SMJP1 (e.g. RemoteDiagnosis) is a parent SMJP of both SMJP2 and SMJP3 and the SMS Requestor (e.g. User) have already discovered SMJP1 with smjpID_1. In addition, SMJP2 and SMJP3 have no dependency relationship in SMJP1. As shown in the FIG. 28, the relatedSMJPs attribute of SMJP1 indicates that the execution request for SMJP3 is not depending on the execution completion of SMJP2.

In this case, the execution request of SMJP2 on SMS Host 122 and SMJP3 on SMS Host 123 may be sent in parallel. As shown in FIG. 29, the procedure for creating a distributed mashup resource with parallel execution without dependency may be detailed as follows. At step 220, The SMJP association relationships between SMJP1 with smjpID_1 and SMJP2 with smjpID_2, SMJP1 with smjpID_1 and SMJP3 with smjpID_3 have already been built among SMS Host 121, SMS Host 122 and SMS Host 123. SMS Requestor has already discovered SMJP1 with smjpID_1. There is no execution dependency between SMJP2 and SMJP3. At step 221, SMS Requestor sends an SMI creation request to SMS Host SMS Host 121. The SMI creation request may include the ID of the applied SMJP which is smjpID, as well as the input parameters defined by the inputDescriptor resource in the applied SMJP. At step 222, after receiving the creation request from step 221, SMS Host 121 may determine if the input smjpID_1 and inputPara from the SMS Requestor are valid; if not valid, it may return an error code; and if they are valid, it may check the SMJP1's relatedSMJPs attribute and find that it may have SMJP association with SMJP2 on SMS Host 122 and SMJP3 on SMS Host 123. The SMJP2 and SMJP3 may have no dependency relationship and the execution of SMJP2 and SMJP3 may be in parallel. SMS Host 121 knows that the intermediate mashup results from SMS Host 122 based on SMJP2 and SMS Host 123 based on SMJP3 may be calculated at the same time. Then, SMS Host 121 may calculate the final mashup result based on the intermediate mashup results from SMS Host 122 and SMS Host 123, respectively. At step 223, SMS Host 121 may send a SMI discovery or creation request with parameters smjpID_2, inputPara1, maintenancePolicy1 to SMS Host 122 based on the SMJP1 with smjpID_1. Similarly, SMS Host 121 may also send a SMI discovery or creation request with parameters smjpID_3, inputPara2, maintenancePolicy2 to SMS Host 123 based on the SMJP1 with smjpID_1.

With continued reference to FIG. 28, at step 224, in step 224(a), SMS Host 122 may discover if there is an existing SMI2 satisfying the given smjpID_2 and inputPara1. If yes, add the maintenancePolicy1 to its maintenance policy list which may determine how the future changes of SMI2 may be reported to Parent SMS Host 121, and go to Step 226(a); otherwise go to Step 225(a). Similarly, in Step 224(b), SMS Host 123 discovers if there is an existing SMI3 satisfying the given smjpID_3 and inputPara2. If yes, add the maintenancePolicy2 to its maintenance policy list which may determine how the future changes of SMI3 may be reported to the parent SMS Host 121, go to Step 226(b); otherwise go to Step 225(b). Note that each SMI resource may have a maintenance policy list which stores policies related to how to maintain this SMI resource (e.g., any actions to be taken if one of mashup members of SMI resource becomes unavailable or unreachable). At step 225, based on the fact that no existing SMI2 satisfying the given smjpID_2 and inputPara are found in Step 224(a), SMS Host 122 may create SMI2 based on the parameters given in Step 223(a) such as smjpID_2, inputPara1, and the received maintenancePolicy1 in Step 225 (a). In order to fully create SMI2, SMS Host 122 may still need to discover or determine mashup members of SMI2 based on the memberFilter information included in SMJP2, which is denoted by smjpID_2. If SMI2 is successfully created, SMS Host 122 may return its URI which is the value of smiID_2 to SMS Host 121; otherwise it may respond with error information to SMS Host 121. Similarly, in Step 225(b), SMS Host 123 may create SMI3 based on the inputPara2, SMJP3 with smjpID_3, and the received maintenancePolicy2. If SMS Host 123 creates SMI3, it returns its URI which is the value of smiID_3; otherwise it responds with error information to SMS Host 121.

At step 226, SMS Host 122 may respond with the URI of SMI2 which is smiID_2 if the SMI2 has been created in Step 225 (a); otherwise it may return error information to SMS Host 121. Similarly, SMS Host 123 may respond with the URI of SMI3 which is smiID_3 if the SMI2 has been successfully created in Step 225 (b); otherwise it may return error information to SMS Host 121. At step 227, based on the successful creation of SMI2 and SMI3, SMS Host 121 may create SMI1 with smiID_1 based on those parameters such as smjpID_1 and inputPara. Since there may be no dependency between SMI2 and SMI3, if at least one SMI (e.g. SMI2) is returned, then SMI1 may still be created based on the returned SMI. If SMI1 is successfully created, SMS Host 121 may return the URI of SMI1 to the SMS Requestor 120; otherwise, it returns an error code to the SMS Requestor 120. At step 228, SMS Host 121 responds the URI of SMI1 that may be the value of smiID_1 to the SMS Requestor 120 if SMI1 is successfully created; otherwise, SMS Host 121 may return an error code to the SMS Requestor 220 if the creation of SMI1 is failed.

Note that if one child SMI has not been successfully created, SMS Host 121 may take one of the following actions, potentially dependent on the relatedSMJP of Hierarchical SMJP. Action 1—SMS Host 121 simply uses successfully created child SMIs to create parent SMI. Action 2—SMS Host 121 discards successfully created child SMIs and stops creating parent SMI. Action 3—SMS Host 121 attempts to discover other SMS Hosts to replace the SMS Host where the child SMI has not been successfully created.

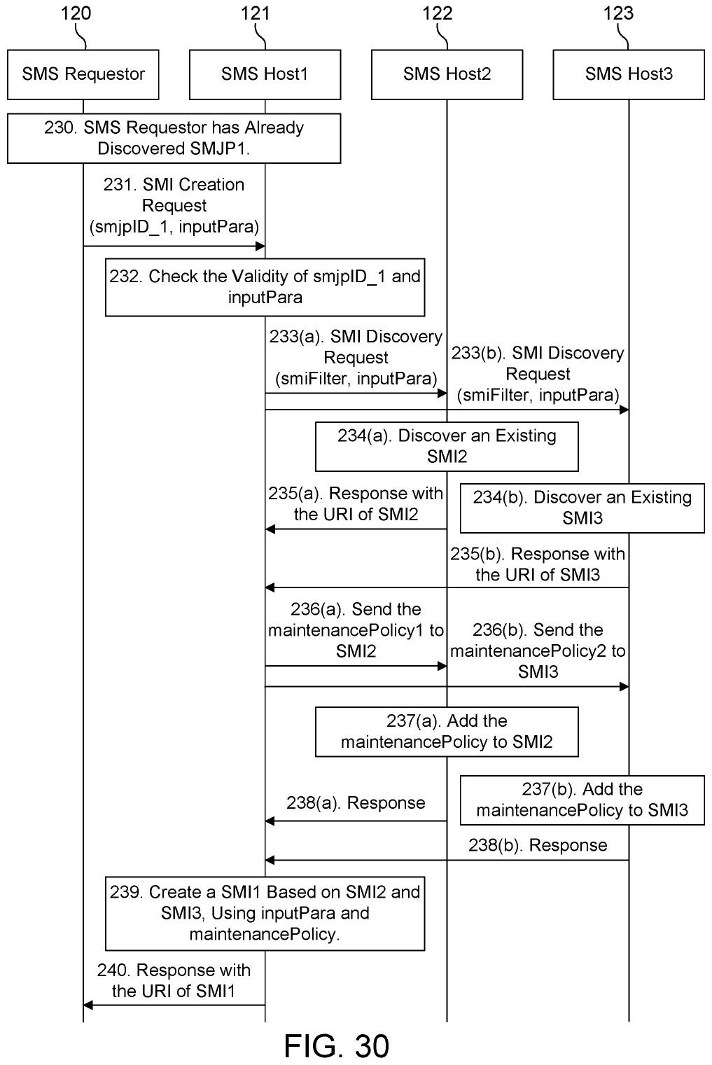

Distributed Semantic Mashup for Hierarchical SMI Generation without SMJP Association—Considering the Smart eHealth use case herein, Server 106 in Clinic A may send diagnosis discovery requests to Server 107 in Hospital B and Server 108 in Hospital C to see if they already have the diagnosis results with the same symptom information matching the symptom information included in the discovery requests. Normally in this scenario, the discovery requests may be sent in parallel. Note: This approach may be seen as an alternative approach without SMJP association compared with the previous approaches using SMJP association for SMI creation. It may be assumed that SMS Host 122 and SMS Host 123 have existing SMIs that satisfy the discovery request from SMS Host 121. As shown in FIG. 30, the procedure for creating distributed mashup instances using semantic discovery without SMJP association may be detailed as follows. At step 230, SMS Host 121 has an SMJP1 with smjpID_1 in its SMJP library, SMS Host 122 has an SMJP2 with smjpID_2 in its SMJP library and SMS Host 123 has an SMJP3 with smjpID_3 in its SMJP library. SMS Requestor has already discovered SMJP1 with smjpID_1 from SMS Host 121. SMJP1 is not associated with either SMJP2 or SMJP3. At step 231, SMS Requestor 120 may send an SMI creation request to SMS Host 121. The SMI creation request may include the ID of the applied SMJP which is smjpID, as well as the input parameters inputPara defined by the inputDescriptor resource in the applied SMJP. At step 232, after receiving the creation request from step 231, SMS Host 121 checks if the input smjpID_1 and inputPara from the SMS Requestor 120 are valid; if not valid, it returns an error code; and if they are valid, it may prepare the SMI discovery request to all registree SMS Hosts which are SMS Host 122 and SMS Host 123. At step 233, SMS Host 121 sends an SMI discovery request to SMS Host 122. Similarly, SMS Host 121 also may send a SMI discovery request SMS Host 123. The SMI discovery request include smiFilter in order to discover related SMIs and also inputPara for using the discovered SMIs. smiFilter describes which types of SMIs may be discovered, while inputPara serves as input parameters from a SMS Requestor 120 in order to use the discovered SMI to generate semantic mashup result. Without giving inputPara to the discovered SMI, it cannot generate correct semantic mashup result as expected by SMS Host 121.

At step 234, in Step 234(a), SMS Host 122 discovers if there is an existing SMI2 satisfying the smiFilter. If yes, the URI of SMI2 which is smiID_2 may be returned to SMS Host 121; otherwise an error code may be returned to SMS Host 121 which means there is no qualified SMIs satisfying the discovery request. Similarly, in Step 234(b), SMS Host 123 may discover if there is an existing SMI3 satisfying the smiFilter. If yes, the URI of SMI3 which is smiID_3 may be returned to SMS Host 121; otherwise an error code may be returned to SMS Host 121 which may mean there is no qualified SMIs satisfying the discovery request. At step 235, SMS Host 122 responds with the URI of SMI2 which is smiID_2 to SMS Host 121 if the SMI2 has been successfully discovered in Step 234(a); otherwise SMS Host 122 may return error information to SMS Host 121. Similarly, SMS Host 123 may respond with the URI of SMI3 which is smiID_3 if the SMI3 has been successfully discovered in Step 234(b); otherwise SMS Host 123 returns error information to SMS Host 121. At step 236, Upon receiving URIs of the SMI2 and SMI3, SMS Host 121 may send the maintenancePolicy1 to SMS Host 122 for SMI2 and maintenancePolicy2 to SMS Host 123 for SMI3. At step 237, in Step 237(a), after receiving the maintenancePolicy1 from SMS Host 121, SMS Host 122 may add the maintenancePolicy1 to the SMI2's maintenance policy list, which may determine how the future changes of SMI2 may be reported to the master SMS Host 121. Similarly, SMS Host 123 may also add the maintenancePolicy2 to the SMI3's maintenance policy list. At step 238, SMS Host 122 and SMS Host 123 may respond the confirmation information to SMS Host 121. At step 239, After receiving the confirmation information from SMS Host 122 and SMS Host 123, SMS Host 121 may create an SMI1 with smiID_1 based on: 1) member resources which are SMI2 with smiID_2 and SMI3 with smiID_3; 2) input parameters which is inputPara; and 3) the maintenancePolicy. If SMI1 may be created by SMS Host 121, the URI of SMI1 may be returned to SMS Requestor 120; otherwise, an error code may be returned from SMS Host 121 to SMS Requestor 120. At step 240, SMS Host 121 responds the URI of SMI1 which is the value of smiID_1 to the SMS Requestor 120 if SMI1 is successfully created; otherwise, SMS Host 121 may return an error code if SMI1 is failed to be created. The parameter in the message is as follows:

Disclosed below are some architectural configurations and oneM2M resource attributes that may support the disclosed distributed SMS under oneM2M functional architecture. First, an architectural configuration for supporting distributed semantic mashup service under oneM2M functional architecture is exemplified. Second, seven new attributes for oneM2M resources (e.g., <smanticMashupJobProfile>, <semanticMashupInstance> resources) are disclosed. Attribute smsIndication attribute of <CSEBase> and <remoteCSE> resources is disclosed to model and represent the indication of SMS capability. The smsIndication may be used as a new parameter in both an oneM2M REQUEST message and an oneM2M RESPONSE message. relatedSMJPs is disclosed to model and represent the relationships and dependencies of two or more semantic mashup profiles. The parentSMIs and childSMIs attributes are disclosed to represent lists of parent semantic mashup instances and child semantic mashup instances in order to form more powerful Hierarchical SMIs. The maintenancePolicy attribute is disclosed to represent a list of policies for maintaining the <SMI> when this <SMI> involved in Hierarchical SMI generation processes. Third, an overall procedure example is described for implementing distributed semantic mashup service under oneM2M functional architecture.

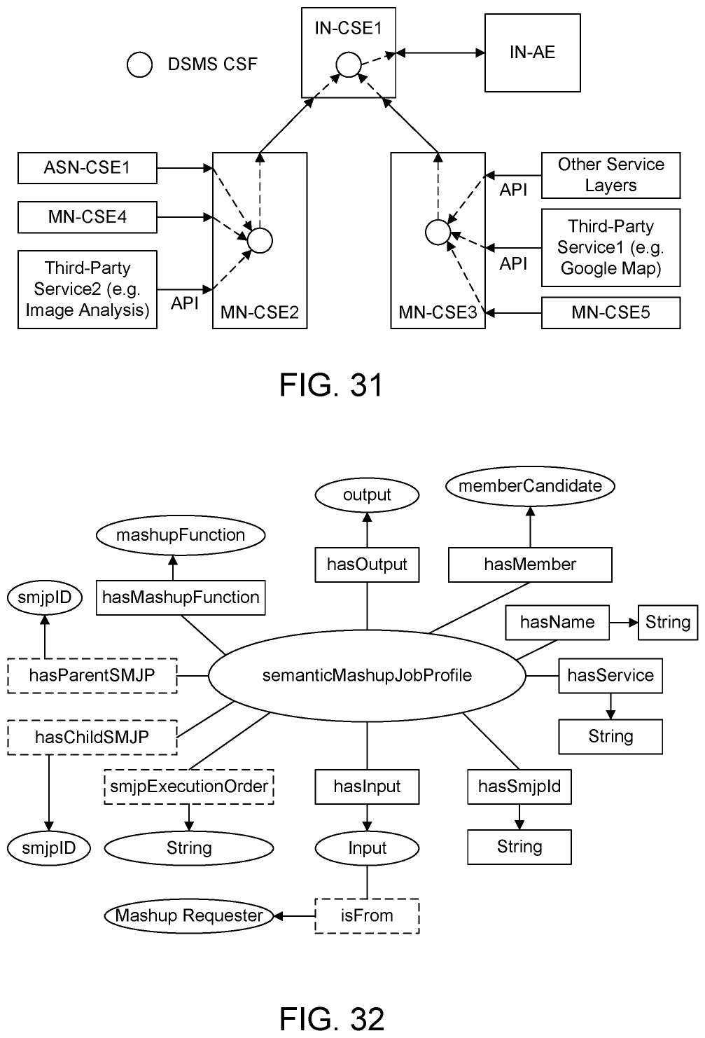

Advanced SMS Configurations in oneM2M Architecture —the Distributed SMS (DSMS) disclosed herein may be implemented under oneM2M functional architecture as a new CSF (e.g. DSMS CSF). This DSMS CSF may be deployed in CSEs (e.g. in FIG. 31). The DSMS CSF may be a realization of functionalities of an SMS Host as described herein. IN-CSE may provide DSMS based on SMI resources from other IN-CSEs, MN-CSEs and ASN-CSEs, which mashup resources from other IN-CSEs, MN-CSEs and ASN-CSEs, services from third-parties, or even services from other service layers. IN-AEs as the SMS requestor 120 may use the DSMS from IN-CSE. Through DSMS, IN-CSE1 leverages SMS CSFs in MN-CSE2 and MN-CSE3 to generate hierarchical SMI. FIG. 31 shows an example where the DSMS CSFs are located in IN-CSE1, MN-CSE2 and MN-CSE3 (note that SMS CSF could also be hosted by ASN/MN-CSE). The DSMS CSF in IN-CSE1 may leverage the SMS CSFs in MN-CSE2 and MN-CSE3 to mashup resources from ASN-CSE1 and MN-CSE4 and MN-CSE5, potentially third-party services, or other service layers, as mashup members to generate hierarchical SMIs. IN-AE may be an SMS Requestor 20 that interfaces to the IN-CSE1 to leverage the SMS CSFs to create hierarchical SMIs based on SMIs from MN-CSE2 and MN-CSE3, to trigger the calculation of the mashup result, or to access the mashup result.

FIG. 32 illustrates an enhanced semantic mashup job profile ontology, where new predicates are disclosed, such as those denoted by the dotted gray rectangles: 1) hasParentSMJP: indicates the Parent SMJP; 2) hasChildSMJP: indicate the Child SMJPs; 3) smjpExecutionOrder: indicate the execution order of all Child SMJPs; or 4) isFrom: indicate the input of a SMJP comes from a mashup requester.

The smsIndication attribute may model and represent the indication of SMS capability of <CSEBase>, <node> and <remoteCSE> resources as described herein. The smsIndication attribute includes two sub-attributes named supportedSMJPs and allowedExternalSMSs. The supportedSMJPs includes a list of URIs and semanticDescriptors of SMJPs of <CSEBase>, <node> and <remoteCSE>. The allowedExternalSMSs includes criteria that may be used to indicate the types of external SMJPs which may be supported by this <CSEBase>, <node> or <remoteCSE>. Alternatively, supportedSMJPs and allowedExternalSMSs may be introduced as two new attributes of <CSEBase>, <node> and <remoteCSE> to replace smsIndication. In addition, the smsIndication may be used as a new parameter in both an oneM2M REQUEST message and an oneM2M RESPONSE message. The smsIndication indicates the SMS capabilities of the oneM2M message senders. This new parameter in oneM2M REQUEST or RESPONSE message enables the SMS discovery, Hierarchical SMJP association, or generation procedures.

The relatedSMJPs attribute is disclosed as to model and represent the relationships and dependencies of two or more semantic mashup job profiles. A relatedSMJPs attribute may be provisioned to the oneM2M CSE which provides semantic mashup service when interact with other oneM2M CSEs which also provide semantic mashup service; alternatively, a special administration application or CSE may request to create or update relatedSMJPs attribute among different oneM2M CSEs. Once relatedSMJPs attributes are provisioned or created with different <semanticMashupJobProfile>s at the oneM2M CSEs (e.g., SMS Hosts), other oneM2M CSEs or AEs, which act as SMS Requestors, may use them via their <semanticMashupJobProfile>s. The relatedSMJPs attribute is specified in Table 1.

TABLE 1
relatedSMJPs attribute of <semanticMashupJobProfile> resource

| Attributes of | |
|---|---|
| <semanticMashup | |
| JobProfile> | Description |
| relatedSMJPs | Semantically describes relationship or |
| | dependency between this <semanticMashupJobProfile> |
| | and other <semanticMashupJobProfile>s (e.g. in |
| | semantic triples). An SMS Requestor do not need to |
| | know this attribute in order to create a |
| | <semanticMashupInstance> based on this semantic |
| | mashup job profile <semanticMashupJobProfile>. |
| | If this <semanticMashupJobProfile> is a Parent |
| | SMJP, this attribute may list all related Child |
| | SMJPs and the execution order to all Child SMJP. |
| | If this <semanticMashupJobProfile> is a Child |
| | SMJP, this attribute only lists the corresponding |
| | Parent SMJP. The value of this attribute may be |
| | in RDF triples or other formats. This attribute is |
| | optional. |

Alternatively, “relatedSMJPs” may be replaced with other two attributes of a <semanticMashupJobProfile>: parentSMJP and childSMJP, in which parentSMJP indicates the parent SMJP of the <semanticMashupJobProfile> resource and childSMJP indicates the child SMJP of the <semanticMashupJobProfile> resource.

The parentSMIs and childSMIs attributes may represent lists of zero or more parent semantic mashup instances and child semantic mashup instances in order to form more powerful Hierarchical SMIs. The parentSMIs and childSMIs attributes may be provisioned to the oneM2M CSE which may provide semantic mashup service when interacting with other oneM2M CSEs which may also provide semantic mashup service; alternatively, a special administration application or CSE may request to create or update parentSMIs and childSMIs attributes among different oneM2M CSEs. Once parentSMIs and childSMIs attributes are provisioned or created with different <semanticMashupInstance>s at the oneM2M CSEs (e.g., SMS Hosts), other oneM2M CSEs or AEs, which act as SMS Requestors 120, may use them via their <semanticMashupInstance>s. Table 2 provides an exemplary description of attributes for <semanticMashupInstance>.

TABLE 2
parentSMIs and childSMIs attributes of <semanticMashupInstance>
resource

| Attributes of | |
|---|---|
| <semanticMashupInstance> | Description |
| parentSMIs | Stands for a list of identifiers of |
| | related semantic mashup instances |
| | which are parent mashup instances of |
| | this <semanticMashupInstance>. |
| childSMIs | Stands for a list of identifiers of |
| | related semantic mashup instances |
| | which are child mashup instances of |
| | this <semanticMashupInstance>. |

The maintenancePolicy attribute may represent a list of policies for maintaining the <semanticMashupInstance> when it is involved in Hierarchical SMI generation process. The maintenancePolicy is a list of policies and related SMS Hosts' IDs preparing for different Parent SMS Hosts or SMS Requestors. The maintenancePolicy attribute may be provisioned to the oneM2M CSE which provides semantic mashup service when interact with other oneM2M CSEs which also provide semantic mashup service; alternatively, a special administration application or CSE may request to create or update maintenancePolicy attribute among different oneM2M CSEs. Once maintenancePolicy attributes are provisioned or created with different <semanticMashupInstance>s at the oneM2M CSEs (e.g., SMS Hosts), other oneM2M CSEs or AEs, which act as SMS Requestors, may use them via their <semanticMashupInstance>s. Table 3 provides an exemplary description of maintenancePolicy.

TABLE 3
maintenancePolicy attribute of <semanticMashupInstance> resource

| Attributes of | |
|---|---|
| <semanticMashupInstance> | Description |
| maintenancePolicy | This attribute indicates this |
| | <semanticMashupInstance> involved in |
| | Hierarchical SMI generation process. |
| | It stands for a list of maintenance |
| | policies and related SMS Host IDs |
| | received from different Parent SMS |
| | Hosts that are serving for those |
| | different Parent SMS Hosts or SMS |
| | Requestors. For maintenancePolicy, it |
| | describes how the mashup result should |
| | be generated using this <semanticMashupInstance>. |
| | Example values for this attribute could |
| | be one of the following or a combination of them. |
| | If maintenancePolicy = “When Child |
| | SMI Is Created”, the semantic mashup |
| | result may be generated when this |
| | <semanticMashupInstance> is created by |
| | running semantic functions specified |
| | by the corresponding <semanticMashupJobProfile>. |
| | If maintenancePolicy = “When Master |
| | SMS Host requests”, the mashup result is |
| | to be calculated and generated when requested |
| | or triggered by an SMS Requestor by sending a |
| | RETRIEVE operation to the child resource mashup |
| | of corresponding semantic result instance. |
| | If maintenancePolicy = “When A Mashup |
| | Member Is Updated”, SMS Host (or SMI |
| | Host) may calculate and generate the |
| | semantic mashup result whenever any |
| | update conducts on the mashup members |
| | of the SMI. |

FIG. 33 illustrates an exemplary overall procedure example for supporting distributed semantic mashup in oneM2M functional architecture. It may include the following parts: SMS Capability Indication and Discovery (e.g., Steps 261-264), Hierarchical SMJP Association and Generation (e.g., Steps 265-269), or Distributed Semantic Mashup for Hierarchical SMI Generation with SMJP Association (e.g. Sequential Execution) (e.g., Steps 270-283). In this example, the following assumptions may be made: 1) IN-AE is an SMS Requestor. IN-CSE acts as a Parent SMS Host and it hosts <semanticMashupJobProfile> resources and <semanticMashupInstance> resources to provide distributed semantic mashup services; or 2) MN-CSE1 and MN-CSE2 act as Child SMS Hosts and host <semanticMashupJobProile> resources, <semanticMashupInstance> resources to provide semantic mashup services to IN-CSE as well as other SMS Requestors.

The overall procedures on FIG. 33 are detailed as follows. At step 261, MN-CSE2 and MN-CSE3 send service layer registration requests to IN-CSE1 with their SMS capability indications. At step 261.a: MN-CSE2 sends a service layer registration request to IN-CSE1 with smsIndication parameter included to indicate its SMS capability. At step 261.b: MN-CSE3 sends a service layer registration request to IN-CSE1 with smsIndication parameter included to indicate its SMS capability. At step 262: IN-CSE1 responds to MN-CSE2 and MN-CSE3 with its smsIndication parameter to indicate its SMS capability. At step 262.a: IN-CSE1 responds to MN-CSE2 with its smsIndication parameter to indicate its SMS capability. At step 262.b: IN-CSE1 responds to MN-CSE3 with its smsIndication parameter to indicate its SMS capability. At step 263: IN-AE initiates semantic discovery with smsFilter in order to discover a list of SMS Hosts that have the interested SMS capabilities. For this example, IN-AE only needs to discover and confirm if IN-CSE supports desired SMSs/SMJPs. At step 264: IN-CSE1 returns the discovered list of SMS Hosts' URIs to IN-AE. At step 265: Based on smsIndications from MN-CSE2 and MN-CSE3, IN-CSE1 learns that SMJP2 (smjpID_2) is a potential child of SMJP1 (smjpID_1) and SMJP3 (smjpID_3) is a potential child of SMJP1 (smjpID_1). IN-CSE1 sends SMJP association requests to MN-CSE2 and MN-CSE3, respectively. At step 265.a: IN-CSE1 sends an SMJP association request with SMJP1's representation which is SMJPRepresentation_1 and SMJP2's URI which is smjpID_2 to MN-CSE2. At step 265.b: IN-CSE1 sends an SMJP association request with SMJP1's representation which is SMJPRepresentation_1 and SMJP3's URI which is smjpID_3 to MN-CSE3. At step 266: After receiving the association requests from IN CSE1 in Step 265, MN-CSE2 and MN-CSE3 may check if SMJP1 and SMJP2, SMJP1 and SMJP3 may be associated based on the contents in these SMJPs. At step 266.a: MN-CSE2 may check if the output parameters in SMJP2's outputDescriptor match the parameters in SMJP1's memberFilter; MN-CSE2 may also check if the mashup metrics in memberFilter of SMJP1 and SMJP2 are matched, and then check if the input parameters in SMJP2's inputDescriptor (if exists) may be provided by the input parameters in SMJP1's inputDescriptor. At step 266.b: MN-CSE3 may check if the output parameters in SMJP3's outputDescriptor match the parameters in SMJP1's memberFilter; MN-CSE3 may also check if the mashup metrics in memberFilter of SMJP1 and SMJP3 are matched, and then check if the input parameters in SMJP3's inputDescriptor (if exists) may be provided by the input parameters in SMJP1's inputDescriptor.

At step 267: After the SMJPs' checking procedures in Step 266, if all matches between SMJP1 and SMJP2 as well as SMJP1 and SMJP3, MN-CSE2 and MN-CSE3 link their SMJPs with SMJP1 in their SMJPs' relatedSMJPs; otherwise MN-CSE2 and MN-CSE3 deny the requests. At step 267.a: If all matches in Step 266.a, MN-CSE2 may create the RDF Triples in SMJP2's relatedSMJPs to link the SMJP2 with SMJP1; otherwise, MN-CSE2 may deny the request. At step 267.b: If all matches in Step 266.b, MN-CSE3 may create the RDF Triples in SMJP2's relatedSMJPs to link the SMJP3 with SMJP1; otherwise, MN-CSE3 may deny the request. At step 268: MN-CSE2 and MN-CSE3 respond the parent-child association confirmation information to IN-CSE1. At step 268.a: MN-CSE2 respond to IN-CSE1 to confirm the SMJP association between SMJP1 and SMJP2. At step 268.b: MN-CSE3 respond to IN-CSE2 to confirm the SMJP association between SMJP1 and SMJP3. At step 269: IN-CSE1 creates RDF Triples in SMJP1's relatedSMJPs to link the SMJP1 with SMJP2 and SMJP3 for parent-child association relationships. At step 269.a: IN-CSE1 creates RDF Triples in SMJP1's relatedSMJPs to link the SMJP1 with SMJP2 for parent-child association relationship. At step 269.b: IN-CSE1 creates RDF Triples in SMJP1's relatedSMJPs to link the SMJP1 with SMJP3 for parent-child association relationship. At step 270: IN-AE sends an <SMI> resource creation request to IN-CSE1. The <SMI> creation request may include the ID of the applied SMJP (e.g., the value of smjpID), as well as the input parameters defined by inputDescriptor. For instance, the parameters in the message sent to IN-CSE1 are as follows:
smjpID_1: the URI of the Master SMJP1 on SMS Host 121.
inputPara: the input parameters that are defined and described by the inputDescriptor resource in the applied SMJP1 as denoted by smjpID_1.

At step 271: After receiving the <SMI> creation request from Step 270, IN-CSE1 checks if the inputs smjpID_1 and inputPara from IN-AE are valid. If they are not valid, it returns error code to IN-AE; if they are valid, it then checks the SMJP1's relatedSMJPs attribute and finds that SMJP1 has SMJP associations with SMJP2 on MN-CSE2 and SMJP3 on MN-CSE3. Also, there is a dependency between SMJP2 and SMJP3 (e.g. the execution of SMJP3 depends on the execution completion of SMJP2 in this example). According to this dependency, IN-CSE1 knows that the <SMI> creation request for SMJP3 must depend on the completion of <SMI> creation for SMJP2. At step 272: IN-CSE1 sends an <SMI> discovery/creation request with parameters smjpID_2, inputPara1, maintenancePolicy1 to MN-CSE2. The parameters in the message sent to MN-CSE2 are as follows:
smjpID_2: the URI of the SMJP2 on SMS Host 122.
inputPara1: the value of input parameters that are defined and described by the inputDescriptor in the applied SMJP2 with smjpID_2.
maintenancePolicy1: the maintenance policy about how to report to IN-CSE about the future changes about the corresponding SMI that may be discovered/created, e.g. if the child SMI is deleted or updated, the Child SMS Host may report this to the Master SMS Host, etc.

At step 273: After receiving the request from IN-CSE1, MN-CSE2 may discover if there is an existing SMI2 satisfying the given smjpID_2 and inputPara1. If yes, add the maintenance policy maintenancePolicy1 to this SMI2's maintenance policy list which may determine how the future changes of SMI2 may be reported to IN-CSE1, go to Step 275; otherwise go to Step 274. Note that each SMI resource has a maintenance policy list which stores policies related how to maintain this SMI resource (e.g. any actions to be taken if one of mashup members of SMI resource becomes unavailable or unreachable). At step 274: Based on the fact that no existing SMI2 satisfying the given smjpID_2 and inputPara is found in Step 273, MN-CSE2 may create an SMI2 based on those parameters given in Step 272 such as smjpID_2, inputPara1, and maintenancePolicy1. In order to fully create SMI2, SMS Host 122 still needs to discover and determine mashup members of SMI2 based on the memberFilter information included in SMJP2, which is denoted by smjpID_2. If SMI2 is successfully created, MN-CSE2 returns its URI which is the value of smiID_2 to IN-CSE1; otherwise, MN-CSE2 responds with error code to IN-CSE1. At step 275: MN-CSE2 responds to IN-CSE1 with the URI of SMI2 which is the value of smiID_2 if the SMI2 has been successfully discovered or created; otherwise it returns error code to IN-CSE1. The parameter in the message sent to SMS Host 122 is as follows:
smiID_2: the URI of the SMI2 on SMS Host 122; or error code.

At step 276: IN-CSE1 checks if the MN-CSE2 has successfully discovered/created SMI2 and returned its URI which is smiID_2. It SMI2's URI has been returned, go to Step 277; otherwise, due to SMJP3's execution dependency on SMJP2 as described in the <relatedSMJPs> of SMJP1 with smjpID_1, the SMI discovery/creation request to MN-CSE3 and subsequent SMI3's discovery/creation may be cancelled, and go to Step 283. At step 277: IN-CSE1 sends an SMI discovery/creation request with parameters smjpID_3, inputPara2, and maintenancePolicy2 to MN-CSE3. The parameters in the message sent to MN-CSE3 are as follows:
smjpID_3: the URI of the SMJP3 on SMS Host 123;
inputPara2: the input parameters that are defined and described by the inputDescriptor in the applied SMJP3 with smjpID_3;
maintenancePolicy2: the maintenance policy about how to report to the master SMS Host 121 about the future changes about the corresponding SMI that may be discovered/created.

At step 278: After receiving the request from IN-CSE1, MN-CSE3 may discover if there is an existing SMI3 satisfying the given smjpID_3 and inputPara2. If yes, add the maintenance policy maintenancePolicy2 to this SMI3's maintenance policy list which may determine how the future changes of SMI3 may be reported to IN-CSE1, go to Step 275; otherwise go to Step 274. Note that each SMI resource has a maintenance policy list which stores policies related how to maintain this SMI resource (e.g. any actions to be taken if one of mashup members of SMI resource becomes unavailable or unreachable). At step 279: Based on the fact that no existing SMI3 satisfying the given smjpID_3 and inputPara is found in Step 278, MN-CSE3 may create an SMI3 based on those parameters given in Step 277 such as smjpID_3, inputPara, and maintenancePolicy. In order to fully create SMI3, SMS Host 122 still needs to discover and determine mashup members of SMI3 based on the memberFilter information included in SMJP3, which is denoted by smjpID_3. If SMI2 is successfully created, MN-CSE3 returns its URI which is the value of smiID_3 to IN-CSE1; otherwise, MN-CSE3 responds with error code to IN-CSE1. At step 280: MN-CSE3 responds to IN-CSE1 with the URI of SMI3 which is the value of smiID_3 if the SMI2 has been successfully discovered or created; otherwise it returns error code to IN-CSE1. The parameter in the message sent to SMS Host 122 is as follows:
smiID_3: the URI of the SMI3 on SMS Host 123; or error code.

At step 281: IN-CSE1 checks if the MN-CSE3 has successfully discovered/created SMI2 and returned its URI which is smiID_3. It SMI3's URI has been returned, go to Step 282; otherwise, due to SMJP3's execution dependency on SMJP2 as described in the relatedSMJPs of SMJP1 with smjpID_1, the SMI1's creation may be cancelled, and go to Step 283. At step 282: Based on the successful discovery or creation of SMI2 and SMI3, IN-CSE1 may create an SMI1 with smiID_1 based on: 1) member resources: SMI2 with smiID_2 and SMI3 with smiID_3; 2) input parameters: inputPara; and 3) the maintenancePolicy. If SMI1 is successfully created, IN-CSE1 returns the URI of SMI1 to IN-AE; otherwise, IN-CSE1 returns error code to IN-AE. At step 283: IN-CSE1 may respond the URI of SMI1 which is the value of smiID_1 to the IN-AE if SMI1 is successfully created; otherwise, IN-CSE1 returns error code to IN-AE if the creation of SMI1 is failed. The parameter in the message is as follows:
smiID_1: the URI of the SMI1 on IN-CSE1; or an error code indicating the SMI1's creation failure.

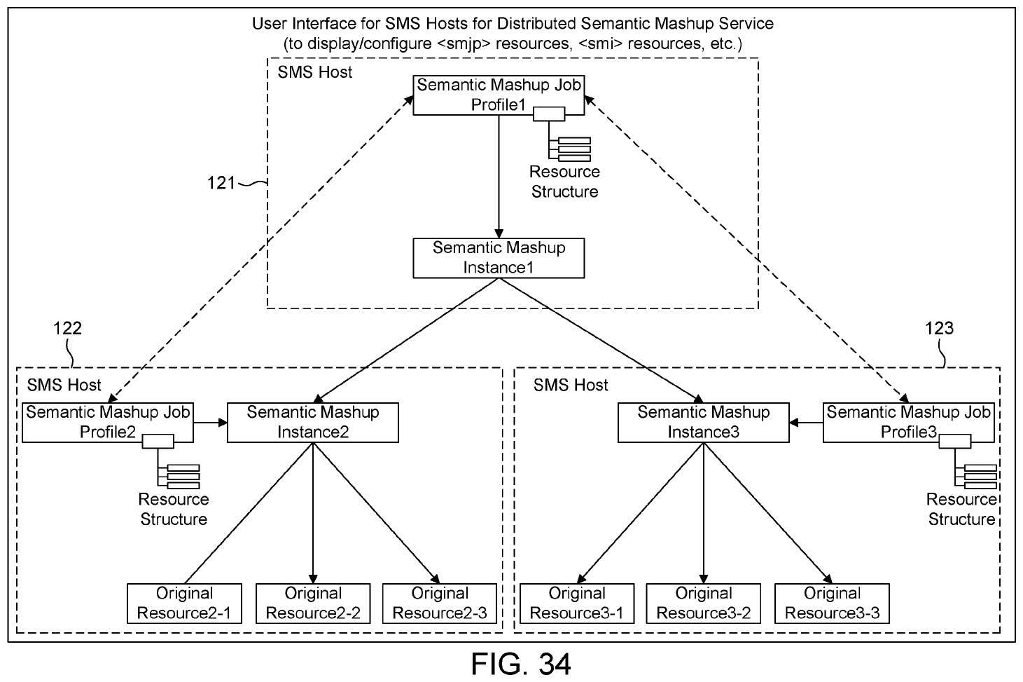

FIG. 34 illustrates a user interface example for SMS Hosts, which may be used to display or configure relationship among semantic mashup profiles (e.g., <semanticMashupJobProfile> resources), semantic mashup resources (e.g., <semanticMashupinstance> resources), and original resources. The resource structure of each semantic mashup job profile in each SMS Host may be displayed or configured. Hierarchical semantic mashup job profile using the different child semantic mashup job profiles from different SMS Hosts may be displayed or configured. For example, it is shown that Hierarchical Semantic Mashup Job Profile1 is using Semantic Mashup Job Profile2 from SMS Host 122 and Semantic Mashup Job Profile3 from SMS Host 123 as Child SMJPs. Hierarchical semantic mashup instance using the different child semantic mashup instances from different SMS Hosts may be displayed or configured. For example, it is shown that Hierarchical Semantic Mashup Instance1 is using Semantic Mashup Instance2 from SMS Host 122 and Semantic Mashup Instance3 from SMS Host 123 as Child SMIs.

Semantic mashup instances using the same semantic mashup job profile may be displayed or configured. For example, it is shown that Semantic Mashup Job Profile2 is used by Semantic Mashup Instance2. Semantic Mashup Job Profile (e.g., Semantic Mashup Job Profile3) is used by one or more semantic mashup instances (e.g., Semantic Mashup Instance3). Member resources of each semantic mashup instance may be displayed or configured. For example, Semantic Mashup Instance2 has three member resources (e.g., Original Resource2-1, Original Resource2-2 and Original Resource2-3); Semantic Mashup Instance3 also has three member resources (e.g., Original Resource3-1, Original Resource3-2 and Original Resource3-3). Through this interface, a member resource may be removed from or a new member resource may be added to a semantic mashup instance.

Without in any way unduly limiting the scope, interpretation, or application of the claims appearing herein, a technical effect of one or more of the examples disclosed herein is to provide adjustments to how to efficiently enable distributed semantic mashup, which may be for an IoT service.

Table 4 provides for exemplary acronym used herein and Table 5 and Table 6 provide for exemplary descriptions of the terminology used herein.

TABLE 4

| Acronym | Definition |
|---|---|
| ACP | Access Control Policy |
| ADN | Application Dedicated Node |
| AE | Application Entity |
| API | Application Program Interface |
| ASN | Application Service Node |
| CRUDN | Create, Retrieve, Update, Delete, Notify |
| CSE | Common Services Entity |
| CSF | Common Services Function |
| HTTP | Hypertext Transfer Protocol |
| IN | Infrastructure Node |
| IN-CSE | CSE which resides in the Infrastructure Node |
| IoT | Internet of Things |
| IRI | Internationalized Resource Identifier |
| M2M | Machine to Machine |
| MN | Middle Node |
| MN-CSE | CSE which resides in the Middle Node |
| NSE | Network Service Entity |
| OWL | Web Ontology Language |
| RDF | Resource Description Framework |
| RDFS | Resource Description Framework Schema |
| SD | Semantic Descriptor |
| SGS | Semantic Graph Store |
| SMI | Semantic Mashup Instance |
| SMJP | Semantic Mashup Job Profile |
| SMR | Semantic Mashup Result |
| SMS | Semantic Mashup Service |
| SPARQL | SPARQL Protocol and RDF Query Language |
| SQL | Structured Query Language |
| Turtle | Terse RDF Triple Language |
| URI | Uniform Resource Identifier |
| W3C | World Wide Web Consortium |
| XHTML | Extensible HTML |
| XML | extensible Markup Language |

TABLE 5

| Terms | Description |
|---|---|
| <semanticDescriptor> | A type of oneM2M resources, which is |
| | usually added as a child resource of |
| | another oneM2M parent resource (e.g., |
| | <contentInstance>) to describe its semantic |
| | information or metadata as RDF triples. |
| Semantic Mashup | A process to discover and collect data |
| | from more than one source as inputs, |
| | conduct a kind of business logic-related |
| | mashup function over the collected data, and |
| | eventually generate meaningful mashup results. |
| Mashup Function | For a given semantic mashup process, after |
| | the SMF collects data inputs from the |
| | qualified data sources, the next step is |
| | to mashup those inputs and derive the |
| | mashup result, which is described by the |
| | mashup function. |
| Semantic Mashup Service | A type of service providing high-level |
| (SMS) | knowledge based on the Semantic Mashup process. |
| | An SMS can be requested by SMS Requestors; |
| | also, an SMS exposes the generated Semantic |
| | Mashup Result (e.g., high-level knowledge) |
| | to SMS Requestors. |
| Semantic Data Model | Semantic Data Model is a method of structuring |
| | data in order to represent it in a specific |
| | logical way. It is a conceptual data model |
| | that includes semantic information that adds |
| | a basic meaning to the data and the relationships |
| | that lie between them. In this paper, we apply |
| | Resource Description Framework (RDF) triples |
| | as Semantic Data Model. |
| Semantic Mashup Job Profile | Each specific semantic mashup service or |
| (SMJP) | application has a corresponding SMJP, which |
| | not only provides functionality/interaction |
| | details for external entities to discover |
| | (e.g. SMS Requestors), but also defines the |
| | internal working details regarding how to |
| | realize this mashup application (e.g. the |
| | criteria of how to select the qualified data |
| | sources as well as the definition of |
| | mashup function). |
| Semantic Mashup Result | The result generated by the Semantic Mashup |
| (SMR) | process. A SMS is described by a semantic data |
| | model (e.g., RDF triples) and may be retrieved |
| | by an SMS Requestor. |
| Semantic Mashup Instance | Once an SMS Requestor identifies a desired |
| (SMI) | SMJP (which may be analogous to a “job |
| | description”, but not a real job), it may |
| | ask SMS host to initialize a real mashup process |
| | (which corresponds to a “working instance” |
| | of this SMJP and is called Semantic Mashup |
| | Instance, or SMI. |
| SMS Host | SMS Host is a logical or physical entity, |
| | which has the ability to provide SMS to SMS |
| | Requestors. For example, an oneM2M CSE could |
| | be an SMS Host. |
| SMS Requestor | SMS Requestor is a logical or physical entity, |
| | which requests and uses/consumes SMSs. An SMS |
| | Requestor may discover (or even create) SMJPs, |
| | create/discover/access SMIs according to certain |
| | SMJPs, and access SMIs. For example, an |
| | oneM2M CSE/AE could be an SMS Requestor. |
| SMJP Host | SMJP Host is a logical or physical entity, |
| | which includes a set of SMJP resources. For |
| | example, an oneM2M CSE could be an SMJP Host. |
| SMI Host | SMI Host is a logical or physical entity, which |
| | includes a number of SMIs. For example, an oneM2M |
| | CSE could be a SMI Host. |
| SMRHost | SMR Host is a logical or physical entity, which |
| | includes a set of SMR resources. For example, an |
| | oneM2M CSE could be an SMR Host. |
| Resource Host | A logical or physical entity, which includes a |
| | set of original resources to be leveraged by the |
| | Semantic Mashup process to calculate SMI. For |
| | example, an oneM2M CSE could be a Resource Host. |
| Parent SMI | A type of higher level SMI that leverages other |
| | SMIs as its member resources during its creation. |
| Child SMI | A type of SMIs that are used by some Parent SMI |
| | as member resources. |
| Hierarchical SMI | An SMI that resides on the Master SMS Host that |
| | directly requested by SMS Requesters and has at |
| | least one or more than one child SMIs. . |
| Master SMS Host | An SMS Host with the role that directly handles |
| | the SMS requests from SMS Requestors and leverages |
| | other SMS Hosts to generate a Hierarchical SMI to |
| | satisfy the SMS request. This role may be temporary |
| | and may be switched to other role in some cases. |
| Child SMS Host | An SMS Host that handles SMS requests from other |
| | SMS Hosts to generate SMIs which may be used as a |
| | member resources for those SMS Hosts. This role may |
| | be also temporary and may be switched to other role |
| | in some cases. |
| Parent SMJP | An SMJP that has other SMJPs as its children which |
| | means when using this SMJP to generate an SMI, it has |
| | some connected SMJPs that may be used to generate |
| | related SMIs which may be used as member resources |
| | for the SMI based on this SMJP. Two SMJPs may be |
| | formulated as a Parent-Child relationship when they |
| | match certain criteria (e.g., the mashup result of |
| | Child SMJP matches the member filter of Parent SMJP). |
| Child SMJP | A type of SMJP that has some Parent SMJPs which |
| | may use this SMJP to generate SMI that may be used |
| | as member resources of SMI based on the Parent |
| | SMJPs. Two SMJPs may be formulated as a Parent- |
| | Child relationship when they match certain criteria |
| | (e.g., the mashup result of Child SMJP matches the |
| | member filter of Parent SMJP). |
| Hierarchical SMJP | An SMJP that resides on the Master SMS Host that |
| | directly requested by SMS Requesters and has at least |
| | one or more than one child SMJP. Any SMJPs that has |
| | Child SMJPs may become Hierarchical SMJPs when |
| | they are directly requested by SMS Requestors. |
| Final Mashup Result | The mashup result generated by the Hierarchical SMI |
| | that resides on the Master SMS Host that directly |
| | received the SMS requests. |
| Intermediate Mashup Result | The mashup result generated by the SMI that resides on |
| | a Child SMS Host which may be used as the input |
| | when calculating the Final Mashup Result. |
| Distributed Semantic Mashup | The existing semantic mashup function as defined in |
| Services (DSMS) | oneM2M is a centralized approach, which extracts all |
| | the original resources to a centralized SMS Host for |
| | mashup operations. In contrast, DSMS leverages a |
| | group of SMS Hosts to conduct mashup operations in a |
| | distributed manner (e.g., each involved Child SMS |
| | Host may conduct certain mashup operations locally on |
| | its own) to generate Intermediate Mashup Results, |
| | which may be used as the member resources of a |
| | Parent SMI residing on Master SMS Host. The Master |
| | SMS Host eventually conducts mashup operations and |
| | generates Final Mashup Result based on those |
| | Intermediate Mashup Results, and returns to SMS |
| | Requestors. |

TABLE 6

| Terms | Description |
|---|---|
| memberFilter | Semantically describes (e.g., in semantic triples) |
| | the types of member resources, which may be involved |
| | in a semantic mashup profile. When a virtual semantic |
| | mashup resource is created based on the semantic mashup |
| | profile, the member resources of the virtual semantic |
| | mashup resource may be discovered and selected based |
| | on this memberFilter attribute. |
| inputDescriptor | Semantically may describe (e.g., in semantic triples) |
| | the types of parameters that may be required as input |
| | parameters in order to use a semantic mashup profile. |
| | An SMS Requestor understand multiple types of input |
| | parameters as described in this attribute in order to |
| | create a virtual semantic mashup resource based on the |
| | semantic mashup profile. |
| outputDescriptor | Semantically may describe (e.g., in semantic triples) |
| | the types of output parameters, which may be generated |
| | as semantic mashup results if using a semantic mashup |
| | profile. |
| functionDescriptor | Semantically may describe the mashup function of a |
| | semantic mashup profile. The mashup function of each |
| | semantic mashup profile specifies how semantic mashup |
| | results should be generated based on input parameters |
| | or original member resources, which may be defined by |
| | the memberFilter attribute of semantic mashup profile. |

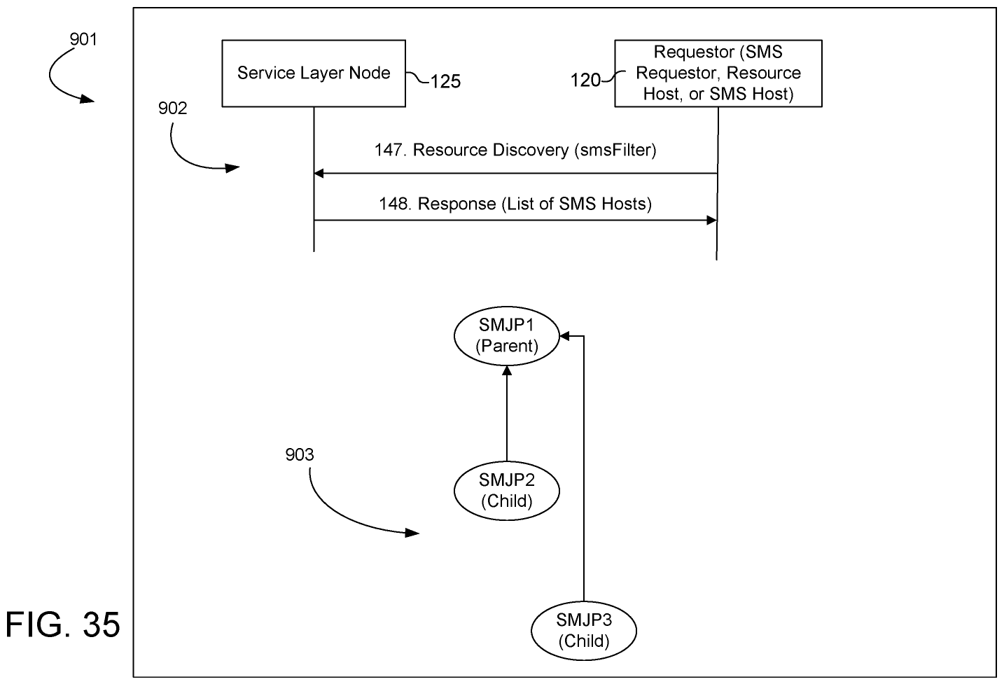

FIG. 35 illustrates an exemplary display (e.g., graphical user interface) that may be generated based on the methods and systems discussed herein. Display interface 901 (e.g., touch screen display) may provide text in block 902 associated with enabling distributed semantic mashup in IoT service layer, such as the parameters of Table 1 through Table 3. In another example, progress of any of the steps (e.g., sent messages or success of steps) discussed herein may be displayed in block 902. In addition, graphical output 903 may be displayed on display interface 901. Graphical output 903 may be the topology of the devices in a graphical output or the progress of any method or systems discussed herein, or the like

FIG. 36A is a diagram of an example machine-to machine (M2M), Internet of Things (IoT), or Web of Things (WoT) communication system 10 in which one or more disclosed concepts associated with enabling distributed semantic mashup in IoT service layer may be implemented (e.g., FIG. 5-FIG. 34 and accompanying discussion). Generally, M2M technologies provide building blocks for the IoT/WoT, and any M2M device, M2M gateway or M2M service platform may be a component of the IoT/WoT as well as an IoT/WoT service layer, etc.

As shown in FIG. 36A, the M2M/IoT/WoT communication system 10 includes a communication network 12. The communication network 12 may be a fixed network (e.g., Ethernet, Fiber, ISDN, PLC, or the like) or a wireless network (e.g., WLAN, cellular, or the like) or a network of heterogeneous networks. For example, the communication network 12 may comprise of multiple access networks that provides content such as voice, data, video, messaging, broadcast, or the like to multiple users. For example, the communication network 12 may employ one or more channel access methods, such as code division multiple access (CDMA), time division multiple access (TDMA), frequency division multiple access (FDMA), orthogonal FDMA (OFDMA), single-carrier FDMA (SC-FDMA), and the like. Further, the communication network 12 may comprise other networks such as a core network, the Internet, a sensor network, an industrial control network, a personal area network, a fused personal network, a satellite network, a home network, or an enterprise network for example.

As shown in FIG. 36A, the M2M/IoT/WoT communication system 10 may include the Infrastructure Domain and the Field Domain. The Infrastructure Domain refers to the network side of the end-to-end M2M deployment, and the Field Domain refers to the area networks, usually behind an M2M gateway. The Field Domain includes M2M gateways 14 and terminal devices 18. It will be appreciated that any number of M2M gateway devices 14 and M2M terminal devices 18 may be included in the M2M/IoT/WoT communication system 10 as desired. Each of the M2M gateway devices 14 and M2M terminal devices 18 are configured to transmit and receive signals via the communication network 12 or direct radio link. The M2M gateway device 14 allows wireless M2M devices (e.g. cellular and non-cellular) as well as fixed network M2M devices (e.g., PLC) to communicate either through operator networks, such as the communication network 12 or direct radio link. For example, the M2M devices 18 may collect data and send the data, via the communication network 12 or direct radio link, to an M2M application 20 or M2M devices 18. The M2M devices 18 may also receive data from the M2M application 20 or an M2M device 18. Further, data and signals may be sent to and received from the M2M application 20 via an M2M service layer 22, as described below. M2M devices 18 and gateways 14 may communicate via various networks including, cellular, WLAN, WPAN (e.g., Zigbee, 6LoWPAN, Bluetooth), direct radio link, and wireline for example.

Referring to FIG. 36B, the illustrated M2M service layer 22 in the field domain provides services for the M2M application 20 (e.g., SMS requestor 120 or gateway 105), M2M gateway devices 14, and M2M terminal devices 18, and the communication network 12. It will be understood that the M2M service layer 22 may communicate with any number of M2M applications, M2M gateway devices 14, M2M terminal devices 18, and communication networks 12 as desired. The M2M service layer 22 may be implemented by one or more servers, computers, or the like. The M2M service layer 22 provides service capabilities that apply to M2M terminal devices 18, M2M gateway devices 14 and M2M applications 20. The functions of the M2M service layer 22 may be implemented in a variety of ways, for example as a web server, in the cellular core network, in the cloud, etc.

Similar to the illustrated M2M service layer 22, there is the M2M service layer 22′ in the Infrastructure Domain. M2M service layer 22′ provides services for the M2M application 20′ and the underlying communication network 12′ in the infrastructure domain. M2M service layer 22′ also provides services for the M2M gateway devices 14 and M2M terminal devices 18 in the field domain. It will be understood that the M2M service layer 22′ may communicate with any number of M2M applications, M2M gateway devices and M2M terminal devices. The M2M service layer 22′ may interact with a service layer by a different service provider. The M2M service layer 22′ may be implemented by one or more servers, computers, virtual machines (e.g., cloud/computer/storage farms, etc.) or the like.

Referring also to FIG. 36B, the M2M service layer 22 and 22′ provide a core set of service delivery capabilities that diverse applications and verticals may leverage. These service capabilities enable M2M applications 20 and 20′ to interact with devices and perform functions such as data collection, data analysis, device management, security, billing, service/device discovery etc. Essentially, these service capabilities free the applications of the burden of implementing these functionalities, thus simplifying application development and reducing cost and time to market. The service layer 22 and 22′ also enables M2M applications 20 and 20′ to communicate through various networks 12 and 12′ in connection with the services that the service layer 22 and 22′ provide.

In some examples, M2M applications 20 and 20′ may include desired applications that communicate using methods or systems for enabling distributed semantic mashup in IoT service layer, as disclosed herein. The M2M applications 20 and 20′ may include applications in various industries such as, without limitation, transportation, health and wellness, connected home, energy management, asset tracking, and security and surveillance. As mentioned above, the M2M service layer, running across the devices, gateways, and other servers of the system, supports functions such as, for example, data collection, device management, security, billing, location tracking/geofencing, device/service discovery, and legacy systems integration, and provides these functions as services to the M2M applications 20 and 20′.

The distributed semantic mashup of the present application may be implemented as part of a service layer. The service layer is a middleware layer that supports value-added service capabilities through a set of application programming interfaces (APIs) and underlying networking interfaces. An M2M entity (e.g., an M2M functional entity such as a device, gateway, or service/platform that is implemented on hardware) may provide an application or service. Both ETSI M2M and oneM2M use a service layer that may include the distributed semantic mashup of the present application. The oneM2M service layer supports a set of Common Service Functions (CSFs) (e.g., service capabilities). An instantiation of a set of one or more particular types of CSFs is referred to as a Common Services Entity (CSE), which may be hosted on different types of network nodes (e.g., infrastructure node, middle node, application-specific node). Further, the distributed semantic mashup of the present application may be implemented as part of an M2M network that uses a Service Oriented Architecture (SOA) or a resource-oriented architecture (ROA) to access services such as the distributed semantic mashup of the present application.

As disclosed herein, the service layer may be a functional layer within a network service architecture. Service layers are typically situated above the application protocol layer such as HTTP, CoAP or MQTT and provide value added services to client applications. The service layer also provides an interface to core networks at a lower resource layer, such as for example, a control layer and transport/access layer. The service layer supports multiple categories of (service) capabilities or functionalities including a service definition, service runtime enablement, policy management, access control, and service clustering. Recently, several industry standards bodies, e.g., oneM2M, have been developing M2M service layers to address the challenges associated with the integration of M2M types of devices and applications into deployments such as the Internet/Web, cellular, enterprise, and home networks. A M2M service layer may provide applications or various devices with access to a collection of or a set of the above mentioned capabilities or functionalities, supported by the service layer, which may be referred to as a CSE or SCL. A few examples include but are not limited to security, charging, data management, device management, discovery, provisioning, and connectivity management which may be commonly used by various applications. These capabilities or functionalities are made available to such various applications via APIs which make use of message formats, resource structures and resource representations defined by the M2M service layer. The CSE or SCL is a functional entity that may be implemented by hardware or software and that provides (service) capabilities or functionalities exposed to various applications or devices (e.g., functional interfaces between such functional entities) in order for them to use such capabilities or functionalities.

FIG. 36C is a system diagram of an example M2M device 30, such as an M2M terminal device 18 (which may include SMS requestor 120 or gateway 105) or an M2M gateway device 14 (which may include one or more components of FIG. 3, FIG. 4, or FIG. 5), for example. As shown in FIG. 36C, the M2M device 30 may include a processor 32, a transceiver 34, a transmit/receive element 36, a speaker/microphone 38, a keypad 40, a display/touchpad 42, non-removable memory 44, removable memory 46, a power source 48, a global positioning system (GPS) chipset 50, and other peripherals 52. It will be appreciated that the M2M device 30 may include any sub-combination of the foregoing elements while remaining consistent with the disclosed subject matter. M2M device 30 (e.g., server 106, server 107, gateway 105, SMS host 121, SMS Requestor 120, IN-AE 250, MN-CSE 252, IN-CSE 251, and others) may be an exemplary implementation that performs the disclosed systems and methods for enabling distributed semantic mashup in IoT service layer.

The processor 32 may be a general purpose processor, a special purpose processor, a conventional processor, a digital signal processor (DSP), a plurality of microprocessors, one or more microprocessors in association with a DSP core, a controller, a microcontroller, Application Specific Integrated Circuits (ASICs), Field Programmable Gate Array (FPGAs) circuits, any other type of integrated circuit (IC), a state machine, and the like. The processor 32 may perform signal coding, data processing, power control, input/output processing, or any other functionality that enables the M2M device 30 to operate in a wireless environment. The processor 32 may be coupled with the transceiver 34, which may be coupled with the transmit/receive element 36. While FIG. 36C depicts the processor 32 and the transceiver 34 as separate components, it will be appreciated that the processor 32 and the transceiver 34 may be integrated together in an electronic package or chip. The processor 32 may perform application-layer programs (e.g., browsers) or radio access-layer (RAN) programs or communications. The processor 32 may perform security operations such as authentication, security key agreement, or cryptographic operations, such as at the access-layer or application layer for example.

The transmit/receive element 36 may be configured to transmit signals to, or receive signals from, an M2M service platform 22. For example, the transmit/receive element 36 may be an antenna configured to transmit or receive RF signals. The transmit/receive element 36 may support various networks and air interfaces, such as WLAN, WPAN, cellular, and the like. In an example, the transmit/receive element 36 may be an emitter/detector configured to transmit or receive IR, UV, or visible light signals, for example. In yet another example, the transmit/receive element 36 may be configured to transmit and receive both RF and light signals. It will be appreciated that the transmit/receive element 36 may be configured to transmit or receive any combination of wireless or wired signals.

In addition, although the transmit/receive element 36 is depicted in FIG. 36C as a single element, the M2M device 30 may include any number of transmit/receive elements 36. More specifically, the M2M device 30 may employ MIMO technology. Thus, in an example, the M2M device 30 may include two or more transmit/receive elements 36 (e.g., multiple antennas) for transmitting and receiving wireless signals.

The transceiver 34 may be configured to modulate the signals that are to be transmitted by the transmit/receive element 36 and to demodulate the signals that are received by the transmit/receive element 36. As noted above, the M2M device 30 may have multi-mode capabilities. Thus, the transceiver 34 may include multiple transceivers for enabling the M2M device 30 to communicate via multiple RATs, such as UTRA and IEEE 802.11, for example.

The processor 32 may access information from, and store data in, any type of suitable memory, such as the non-removable memory 44 or the removable memory 46. The non-removable memory 44 may include random-access memory (RAM), read-only memory (ROM), a hard disk, or any other type of memory storage device. The removable memory 46 may include a subscriber identity module (SIM) card, a memory stick, a secure digital (SD) memory card, and the like. In other examples, the processor 32 may access information from, and store data in, memory that is not physically located on the M2M device 30, such as on a server or a home computer. The processor 32 may be configured to control lighting patterns, images, or colors on the display or indicators 42 in response to whether the distributed semantic mashup in some of the examples described herein are successful or unsuccessful, or otherwise indicate a status of distributed semantic mashup and associated components. The control lighting patterns, images, or colors on the display or indicators 42 may be reflective of the status of any of the method flows or components in the FIG.'S illustrated or discussed herein (e.g., FIG. 5, 11, 12, 17, 27, 29, 30, or 33, etc.). Disclosed herein are messages and procedures of distributed semantic mashup. The messages and procedures may be extended to provide interface/API for users to request service layer related information via an input source (e.g., speaker/microphone 38, keypad 40, or display/touchpad 42). In an addition example, there may be a request, configure, or query of distributed semantic mashup information, among other things that may be displayed on display 42.

The processor 32 may receive power from the power source 48, and may be configured to distribute or control the power to the other components in the M2M device 30. The power source 48 may be any suitable device for powering the M2M device 30. For example, the power source 48 may include one or more dry cell batteries (e.g., nickel-cadmium (NiCd), nickel-zinc (NiZn), nickel metal hydride (NiMH), lithium-ion (Li-ion), etc.), solar cells, fuel cells, and the like.

The processor 32 may also be coupled with the GPS chipset 50, which is configured to provide location information (e.g., longitude and latitude) regarding the current location of the M2M device 30. It will be appreciated that the M2M device 30 may acquire location information by way of any suitable location-determination method while remaining consistent with information disclosed herein.

The processor 32 may further be coupled with other peripherals 52, which may include one or more software or hardware modules that provide additional features, functionality or wired or wireless connectivity. For example, the peripherals 52 may include various sensors such as an accelerometer, biometrics (e.g., fingerprint) sensors, an e-compass, a satellite transceiver, a sensor, a digital camera (for photographs or video), a universal serial bus (USB) port or other interconnect interfaces, a vibration device, a television transceiver, a hands free headset, a Bluetooth® module, a frequency modulated (FM) radio unit, a digital music player, a media player, a video game player module, an Internet browser, and the like.

The transmit/receive elements 36 may be embodied in other apparatuses or devices, such as a sensor, consumer electronics, a wearable device such as a smart watch or smart clothing, a medical or eHealth device, a robot, industrial equipment, a drone, a vehicle such as a car, truck, train, or airplane. The transmit/receive elements 36 may connect to other components, modules, or systems of such apparatuses or devices via one or more interconnect interfaces, such as an interconnect interface that may comprise one of the peripherals 52.

FIG. 36D is a block diagram of an exemplary computing system 90 on which, for example, the M2M service platform 22 of FIG. 36A and FIG. 36B may be implemented. Computing system 90 (e.g., M2M terminal device 18 or M2M gateway device 14) may comprise a computer or server and may be controlled primarily by computer readable instructions by whatever means such instructions are stored or accessed. Such computer readable instructions may be executed within central processing unit (CPU) 91 to cause computing system 90 to do work. In many known workstations, servers, and personal computers, central processing unit 91 is implemented by a single-chip CPU called a microprocessor. In other machines, the central processing unit 91 may comprise multiple processors. Coprocessor 81 is an optional processor, distinct from main CPU 91, that performs additional functions or assists CPU 91. CPU 91 or coprocessor 81 may receive, generate, and process data related to the disclosed systems and methods for distributed semantic mashup, such as receiving distributed semantic mashup message over the control plane.

In operation, CPU 91 fetches, decodes, and executes instructions, and transfers information to and from other resources via the computer's main data-transfer path, system bus 80. Such a system bus connects the components in computing system 90 and defines the medium for data exchange. System bus 80 typically includes data lines for sending data, address lines for sending addresses, and control lines for sending interrupts and for operating the system bus. An example of such a system bus 80 is the PCI (Peripheral Component Interconnect) bus.

Memory devices coupled with system bus 80 include random access memory (RAM) 82 and read only memory (ROM) 93. Such memories include circuitry that allows information to be stored and retrieved. ROMs 93 generally include stored data that cannot easily be modified. Data stored in RAM 82 may be read or changed by CPU 91 or other hardware devices. Access to RAM 82 or ROM 93 may be controlled by memory controller 92. Memory controller 92 may provide an address translation function that translates virtual addresses into physical addresses as instructions are executed. Memory controller 92 may also provide a memory protection function that isolates processes within the system and isolates system processes from user processes. Thus, a program running in a first mode may access only memory mapped by its own process virtual address space; it may not access memory within another process's virtual address space unless memory sharing between the processes has been set up.

In addition, computing system 90 may include peripherals controller 83 responsible for communicating instructions from CPU 91 to peripherals, such as printer 94, keyboard 84, mouse 95, and disk drive 85.

Display 86, which is controlled by display controller 96, is used to display visual output generated by computing system 90. Such visual output may include text, graphics, animated graphics, and video. Display 86 may be implemented with a CRT-based video display, an LCD-based flat-panel display, gas plasma-based flat-panel display, or a touch-panel. Display controller 96 includes electronic components required to generate a video signal that is sent to display 86.

Further, computing system 90 may include network adaptor 97 that may be used to connect computing system 90 to an external communications network, such as network 12 of FIG. 36A and FIG. 36B.

It is understood that any or all of the systems, methods and processes described herein may be embodied in the form of computer executable instructions (e.g., program code) stored on a computer-readable storage medium which instructions, when executed by a machine, such as a computer, server, M2M terminal device, M2M gateway device, or the like, perform or implement the systems, methods and processes described herein. Specifically, any of the steps, operations or functions described above may be implemented in the form of such computer executable instructions. Computer readable storage media include both volatile and nonvolatile, removable and non-removable media implemented in any method or technology for storage of information, but such computer readable storage media do not include signals per se. As evident from the herein description, storage media should be construed to be statutory subject matter. Computer readable storage media include RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other physical medium which may be used to store the desired information and which may be accessed by a computer. A computer-readable storage medium may have a computer program stored thereon, the computer program may be loadable into a data-processing unit and adapted to cause the data-processing unit to execute method steps associated with distributed semantic mashup when the computer program is run by the data-processing unit.

In describing preferred methods, systems, or apparatuses of the subject matter of the present disclosure —distributed semantic mashup —as illustrated in the Figures, specific terminology is employed for the sake of clarity. The claimed subject matter, however, is not intended to be limited to the specific terminology so selected, and it is to be understood that each specific element includes all technical equivalents that operate in a similar manner to accomplish a similar purpose.

The various techniques described herein may be implemented in connection with hardware, firmware, software or, where appropriate, combinations thereof. Such hardware, firmware, and software may reside in apparatuses located at various nodes of a communication network. The apparatuses may operate singly or in combination with each other to effectuate the methods described herein. As used herein, the terms “apparatus,” “network apparatus,” “node,” “device,” “network node,” or the like may be used interchangeably. In addition, the use of the word “or” is generally used inclusively unless otherwise provided herein.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art (e.g., skipping steps, combining steps, or adding steps between exemplary methods disclosed herein). Such other examples are intended to be within the scope of the claims if they have structural elements that do not differ from the literal language of the claims, or if they include equivalent structural elements with insubstantial differences from the literal languages of the claims. Although disclosed herein are systems and methods for enabling distributed semantic mashup in IoT service layer, it is contemplated that systems other than IoT service layer may be used.

A method, system, computer readable storage medium, or apparatus (e.g., node) has means for receiving a request from an entity to associate one parent SMJP with other child semantic mashup job profiles; examining if these child semantic mashup job profiles match with the parent semantic mashup job profile; linking child profiles and the parent profile if they match with each other; and sending a response to the entity for confirming the establishment of parent-child relationship between child profiles and the parent profile. Node may be an IoT server, gateway, or device. Entity may be an IoT server, gateway, device, or application. Parent profile may be included in the request and child profiles may be locally stored at the node. Parent profile may be locally stored at the node and child profiles may be included in the request. A method, system, computer readable storage medium, or apparatus (e.g., node) has means for receiving a request from an entity to create a parent semantic mashup instance based on a parent semantic job profile; discovering each child semantic mashup job profile associated with the parent profile; contacting each child profile to create a corresponding child semantic mashup instance; receiving a response from each child profile; checking (e.g., determining) if all child semantic mashup instances are successfully created; creating the parent semantic mashup instance based created child instances; and sending a response to the entity to confirm the creation of the parent instance. Each child semantic mashup instance may need to be created sequentially according to certain order as described in the parent profile. Each child semantic mashup instance may need to be created in parallel without any particular order as described in the parent profile. All combinations in this paragraph and the following paragraph are contemplated in a manner that is consistent with the other portions of the detail description

A method for enabling distributed semantic mashup, the method comprising: receiving a request from an entity to associate a parent semantic mashup job profile (SMJP) with a child SMJP, wherein the request comprises one or more parameters of the child SMJP; determining whether the one or more parameters of the child SMJP matches one or more parameters of the parent SMJP; linking the child SMJP and the parent SMJP when the one or more parameters of the child SMJP matches the one or more parameters of the parent SMJP, wherein the linking establishes a parent-child relationship between the child SMJP and the parent SMJP; and sending a response to the entity for confirming the establishment of the parent-child relationship between the child SMJP and the parent SMJP. The one or more parameters of the of the request may include inputDescriptor, outputDescriptor, functionDescriptor, memberFilter, or semanticDescriptor. The determining whether the one or more parameters of the child SMJP matches one or more parameters of the parent SMJP may include determining if output parameters described in an outputDescriptor matches member resources criteria described in a memberFilter of the parent SMJP. The determining whether the one or more parameters of the child SMJP matches one or more parameters of the parent SMJP may include determining that the mashup metrics in memberFilter of the parent SMJP and the mashup metrics in memberFilter of child SMJP match. The determining whether the one or more parameters of the child SMJP matches one or more parameters of the parent SMJP may include determining that the input parameters described in an inputDescriptor of the parent SMJP matches the input parameters described in an inputDescriptor of the child SMJP. The determining whether the one or more parameters of the child SMJP matches one or more parameters of the parent SMJP comprises determining that: the output parameters described in an outputDescriptor matches member resources criteria described in a memberFilter of the parent SMJP; the mashup metrics in memberFilter of the parent SMJP and the mashup metrics in memberFilter of child SMJP match; and the input parameters described in an inputDescriptor of the parent SMJP matches the input parameters described in an inputDescriptor of the child SMJP. The linking may include adding related semantic descriptions. The method steps may be executed on a child SMJP host or a parent SMJP host. All combinations in this paragraph are contemplated in a manner that is consistent with the other portions of the detail description.

1. A method for enabling distributed semantic mashup, the method comprising:
receiving a request from an entity to associate a parent semantic mashup job profile (SMJP) with a child SMJP, wherein the request comprises one or more parameters of the child SMJP;
determining whether the one or more parameters of the child SMJP matches one or more parameters of the parent SMJP;
linking the child SMJP and the parent SMJP when the one or more parameters of the child SMJP matches the one or more parameters of the parent SMJP, wherein the linking establishes a parent-child relationship between the child SMJP and the parent SMJP; and
sending a response to the entity for confirming the establishment of the parent-child relationship between the child SMJP and the parent SMJP.

2. The method of claim 1, wherein the one or more parameters of the of the request comprises input descriptor, output descriptor, function descriptor, member filter, or semantic descriptor.

3. The method of claim 1, wherein the determining whether the one or more parameters of the child SMJP matches one or more parameters of the parent SMJP comprises determining if output parameters described in an output descriptor matches member resources criteria described in a member filter of the parent SMJP.

4. The method of claim 1, wherein the determining whether the one or more parameters of the child SMJP matches one or more parameters of the parent SMJP comprises determining that the mashup metrics in member filter of the parent SMJP and the mashup metrics in member filter of child SMJP match.

5. The method of claim 1, wherein the determining whether the one or more parameters of the child SMJP matches one or more parameters of the parent SMJP comprises determining that the input parameters described in an input descriptor of the parent SMJP matches the input parameters described in an input descriptor of the child SMJP.

6. The method of claim 1, wherein the determining whether the one or more parameters of the child SMJP matches one or more parameters of the parent SMJP comprises determining that:
the output parameters described in an output descriptor matches member resources criteria described in a member filter of the parent SMJP;
the mashup metrics in member filter of the parent SMJP and the mashup metrics in member filter of child SMJP match; and
the input parameters described in an input descriptor of the parent SMJP matches the input parameters described in an input descriptor of the child SMJP.

7. The method of claim 1, wherein the linking comprises adding related semantic descriptions.

8. The method of claim 1, wherein the method steps are executed on a child SMJP host.

9. The method of claim 1, wherein the method steps are executed on a parent SMJP host.

10. (canceled)

11. An apparatus for enabling distributed semantic mashup, the apparatus comprising:
a processor; and
a memory coupled with the processor, the memory storing executable instructions that when executed by the processor cause the processor to effectuate operations comprising:
receiving a request from an entity to associate a parent semantic mashup job profile (SMJP) with a child SMJP, wherein the request comprises one or more parameters of the child SMJP;
determining whether the one or more parameters of the child SMJP matches one or more parameters of the parent SMJP;
linking the child SMJP and the parent SMJP when the one or more parameters of the child SMJP matches the one or more parameters of the parent SMJP, wherein the linking establishes a parent-child relationship between the child SMJP and the parent SMJP; and
sending a response to the entity for confirming the establishment of the parent-child relationship between the child SMJP and the parent SMJP.

12. The apparatus of claim 11, wherein the one or more parameters of the of the request comprises input descriptor, output descriptor, function descriptor, member filter, or semantic descriptor.

13. The apparatus of claim 11, wherein the determining whether the one or more parameters of the child SMJP matches one or more parameters of the parent SMJP comprises determining if output parameters described in an output descriptor matches member resources criteria described in a member filter of the parent SMJP.

14. The apparatus of claim 11, wherein the determining whether the one or more parameters of the child SMJP matches one or more parameters of the parent SMJP comprises determining that the mashup metrics in member filter of the parent SMJP and the mashup metrics in member filter of child SMJP match.

15. The apparatus of claim 11, wherein the determining whether the one or more parameters of the child SMJP matches one or more parameters of the parent SMJP comprises determining that the input parameters described in an input descriptor of the parent SMJP matches the input parameters described in an input descriptor of the child SMJP.

16. A system comprising:
one or more processors; and
memory coupled with the one or more processors, the memory storing executable instructions that when executed by the one or more processors cause the one or more processors to effectuate operations comprising:
receiving a request from an entity to associate a parent semantic mashup job profile (SMJP) with a child SMJP, wherein the request comprises one or more parameters of the child SMJP;
determining whether the one or more parameters of the child SMJP matches one or more parameters of the parent SMJP;
linking the child SMJP and the parent SMJP when the one or more parameters of the child SMJP matches the one or more parameters of the parent SMJP, wherein the linking establishes a parent-child relationship between the child SMJP and the parent SMJP; and
sending a response to the entity for confirming the establishment of the parent-child relationship between the child SMJP and the parent SMJP.

17. The system of claim 16, wherein the one or more parameters of the of the request comprises input descriptor, output descriptor, function descriptor, member filter, or semantic descriptor.

18. The system of claim 16, wherein the determining whether the one or more parameters of the child SMJP matches one or more parameters of the parent SMJP comprises determining if output parameters described in an output descriptor matches member resources criteria described in a member filter of the parent SMJP.

19. The system of claim 16, wherein the determining whether the one or more parameters of the child SMJP matches one or more parameters of the parent SMJP comprises determining that the mashup metrics in member filter of the parent SMJP and the mashup metrics in member filter of child SMJP match.

20. The system of claim 16, wherein the determining whether the one or more parameters of the child SMJP matches one or more parameters of the parent SMJP comprises determining that the input parameters described in an input descriptor of the parent SMJP matches the input parameters described in an input descriptor of the child SMJP.

21. The system of claim 16, wherein the linking comprises adding related semantic descriptions.
